SEQUENCE LISTING (11 sequences)

SEQ ID NO: 1 - DNA, 21 bp, Artificial Sequence
  B7-H3 forward primer
gacagcaaagaagatgatgga

SEQ ID NO: 2 - DNA, 25 bp, Artificial Sequence
  B7-H3 fluorescence resonance energy transfer probe sequence
cctccctacagctcctaccctctgg

SEQ ID NO: 3 - DNA, 19 bp, Artificial Sequence
  B7-H3 reverse primer
acctgtcagagcaggatgc

SEQ ID NO: 4 - DNA, 24 bp, Artificial Sequence
  glyceraldehyde-3-phosphate dehydrogenase (GAPDH) probe
cagcaatgcctcctgcaccaccaa

SEQ ID NO: 5 - DNA, 24 bp, Artificial Sequence
  fluorescence resonance energy transfer probe sequence MART-1
cagaacagtcaccaccaccttatt

SEQ ID NO: 6 - DNA, 25 bp, Artificial Sequence
  fluoresence resonance energy transfer probe sequence MAGE-A3
agctcctgcccacactcccgcctgt

SEQ ID NO: 7 - DNA, 20 bp, Artificial Sequence
  fluorescence resonance energy transfer probe sequence HMW-MAA
aggatcaccgtggctgctct

SEQ ID NO: 8 - DNA, 25 bp, Artificial Sequence
  fluorescence resonance energy transfer probe sequence TRP-2
tcacatcaaggacctgcatttgtta

SEQ ID NO: 9 - DNA, 18 bp, Artificial Sequence
  B7-H3 variant forward primer
agagaagccccacaggag

SEQ ID NO: 10 - DNA, 22 bp, Artificial Sequence
  B7-H3 variant fluoresence resonance energy transfer probe sequence
gaggtccaggtccctgaggacc

SEQ ID NO: 11 - DNA, 18 bp, Artificial Sequence
  B7-H3 variant reverse primer
gctgccagatgaggttga

(19) United States
(12) Patent Application Publication
(10) Pub. No.: US 2011/0206705 A1
(43) Pub. Date: Aug. 25, 2011

(54) B7-H3 AS A BIOMARKER FOR DIAGNOSING THE PROGRESSION AND EARLY LYMPH NODE METASTASIS OF CANCER

(76) Inventor: Dave S.B. HOON, Los Angeles, CA (US)

(21) Appl. No.: 12/979,269

(22) Filed: Dec. 27, 2010

Related U.S. Application Data
(63) Continuation of application No. PCT/US2010/024849, filed on Feb. 20, 2010.
(60) Provisional application No. 61/153,975, filed on Feb. 20, 2009.

(51) Int. Cl. A61K 39/395; C12Q 1/02; G01N 33/574; C12Q 1/68; A61P 35/04

(52) U.S. Cl. 424/178.1; 435/29; 435/7.23; 435/6.12

(57) ABSTRACT

B7H3 is a ligand member of the immunoregulatory family of proteins on immune cells. In one embodiment, a method for diagnosing the progression of cancer with a high propensity of primary tumor metastasis to the lymph node or distant site is provided. Such a method may comprise obtaining a cancer tissue sample from a cancer patient, determining an expression level of B7-H3 present in the tissue sample, and diagnosing the progression of the cancer having a high propensity of primary tumor metastasis to the lymph node or distant site based upon the expression level, wherein an increased expression level correlates with an increased probability of having regional lymph nodes or organ site that are positive for metastases.

### CROSS-REFERENCE TO RELATED APPLICATIONS

This application is a continuation of International Application Number PCT/US2010/024849, filed Feb. 20, 2010, which claims the benefit of U.S. Provisional Application No. 61/153,975, filed Feb. 20, 2009, which is incorporated herein by reference in its entirety.

### STATEMENT OF GOVERNMENT SUPPORT

This invention was made with Government support in part (melanoma only section) under Grant Nos. CA029605 and CA012582 awarded by The National Cancer Institute (NCI) Project II P0 of the National Institutes of Health (NIH). The U.S. government has partial rights in the invention. Breast and other cancer studies are supported by private foundations.

### FIELD OF THE INVENTION

The present invention relates in general to the diagnosis, prognosis, and treatment of cancer. More specifically, the invention provides a method of detecting and isolating B7-H3 (+) tumor cells in body fluids and tumor tissues from melanoma and breast cancer patients, as well as its diagnostic and prognostic applications.

### BACKGROUND OF THE INVENTION

The B7-1 (CD80) and B7-2 (CD86) costimulatory molecules are important in regulating T-cell activation and tolerance of host immune responses (Sharpe & Freeman 2002). The B7 ligand family members are known to have strong immunoregulatory activity by immune cells and more recently by tumor cells (Chapoval et al. 2001; Flies & Chen 2007; Greenwald et al. 2005; Zang & Allison 2007). B7 coregulatory ligands have been shown to regulate T-cell immunity through their respective receptors (Flies & Chen 2007; Greenwald et al. 2005). For example, the binding of B7-1 (CD80) and B7-2 (CD86) to receptor CD28 initiates immunoregulatory signaling pathways that can modify the immune response to cancer and other diseases by inducing production of interleukin-2 and regulating T-cell activity in various immune responses (Greenwald et al. 2005; Flies & Chen 2007; Zang & Allison 2007).

B7-1 and B7-2 molecules also have a coinhibitory function on T-cell-mediated immunity through binding of the receptor cytotoxic T lymphocyte antigen-4 (CTLA-4), a homolog of CD28 expressed on activated T-cells (Flies & Chen 2007; Greenwald et al. 2005; Zang & Allison 2007). The B7 ligand family members and receptors are therefore important immunoregulatory factors in host tumor-immune responses.

B7-H3, a type I transmembrane protein, is a recently identified member of the B7 ligand family (Flies & Chen 2007; Greenwald et al. 2005; Zang & Allison 2007; Hofmeyer et al. 2008; Zang et al. 2003). The role of B7-H3 in immune responses, including tumor immunity, remains controversial and unclear. It is strongly suggested that B7-H3 downregulates T-helper immune responses and suppresses immunity (Suh et al. 2003). It is suggested to play a role in regulating immune responses of CD4⁺ and CD8⁺ T-cells. B7-H3 mRNA expression has also been ascertained by northern blot analysis in several tumor types. Recent studies have shown that tumor cells have utilized cell surface immunoregulatory proteins such as B7 ligands and their function to escape host immune responses (Chapoval et al. 2001; Dong et al. 2002).

B7-H3 was first identified in dendritic cells and activated T-cells (Zang & Allison 2007; Chapoval et al. 2001). B7-H3 has been characterized as having two isoforms: Ig containing two domains; IgV and IgC, and Ig containing four domains: IgV-IgC-IgV-IgC (4IgB7-H3). The latter is not constitutive or detectable in resting immune cells (Castriconi et al. 2004; Chen et al. 2008). The receptor for B7-H3 has not been identified to date, although a potential receptor (TREM (triggering receptor expressed in myeloid cells) like transcript 2) for B7-H3 has recently been suggested in the murine system (Hashiguchi et al. 2008).

B7-H3 expression has been detected in several cancer types, however, its role remains uncertain (Castriconi et al. 2004; Roth et al. 2007; Sun et al. 2006; Wu et al. 2006). No reports have yet revealed the relation between B7-H3 and clinicopathological factors in primary breast cancer or its progression to regional metastasis; or in human cutaneous melanoma. B7-H3 is strongly expressed on the membrane and/or cytoplasm of cells (Roth et al. 2007; Sun et al. 2006; Wu et al. 2006). B7-H3 expression has also been suggested to be related to tumor progression (Roth et al. 2007; Sun et al. 2006). Studies have suggested that B7-H3 expressed by tumors may allow cells to escape immune surveillance and promote immune tolerance (Hofmeyer et al. 2008, Roth et al. 2007).

Recently, the B7 ligand family and its receptors have been studied as therapeutic targets in anti-tumor immunotherapy (Zang & Allison 2007). CTLA-4 is a receptor for B7-1 and B7-2 on activated T-cells (Brunet et al. 1987; Linsley et al. 1991; Freeman et al. 1993; Azuma et al. 1993), and it downregulates the T cell-mediated immune response (Waterhouse et al. 1995; Tivol et al. 1995). Therefore, the blocking CTLA-4 may inhibit cancer progression. At present, two human anti-CTLA-4 monoclonal antibodies have completed phase I/II clinical trials in melanoma patients (Hodi et al. 2003; Ribas et al 2005; Beck et al. 2006; O'Mahony et al. 2007). The trials have shown encouraging results and the antibodies are being further examined in clinical phase III trials. Similarly, if B7-H3 serves as a negative regulator for T cell-mediated immune responses, blocking B7-H3 may provide a new approach of targeted therapy similar to anti-CTLA-4 monoclonal antibody therapy. B7-H3 is a different molecule than B7-H1 and H2 and with different functions and ligands.

Melanomas of intermediate and advanced stage have a poor 5-year prognosis, whereby surgery still remains the first line of treatment (Balch et al. 2001). The 5-year survival rates of patients with American Joint Committee on Cancer (AJCC) stage III and IV cutaneous melanomas are 45% and 10%, respectively (Balch et al. 2001). It is difficult to determine risk of recurrence after surgery for early-stage disease. Better prognostic biomarkers would improve risk assessment, however melanoma cell-surface markers of prognostic utility are very limited. Recent studies have shown that immunoregulatory molecules on immune cells can also be expressed on human tumor cells (Flies & Chen 2007; Goto et al. 2008; Greenwald et al. 2005; Zang & Allison 2007). These hijacked immunoregulatory molecules on tumors can downregulate immune responses thus allowing the tumors to escape host tumor immunity. This event may be an important disease progression factor that has previously been ignored.

Early detection of breast cancer often has a good prognosis, but some primary tumors are more aggressive and are elusive to host immune responses. Identification of high-risk patients for potential regional nodal metastasis at time of primary tumor diagnosis would help make decisions on the level of nodal surgical procedure needed. Currently, early stage breast cancer sentinel lymph node (SLN) biopsy can identify patients with lymph node metastasis (Giuliano et al. 1994; Olson et al. 2008; Turner et al. 2008). The procedure can reliably identify patients who have axillary nodal metastasis. However, improvement is needed in identifying primary tumor prognostic factors to identify those patients who will have more aggressive nodal disease. Although several biomarkers of primary breast cancer have been investigated, as yet none have been validated for determining the risk of regional nodal metastasis.

Patients diagnosed with early stage breast cancer potentially have a good prognosis. However, it is difficult to determine which patients will have aggressive disease and spread to the regional nodal basin or will be likely to develop recurrence. As primary breast cancer is being diagnosed earlier, it is becoming more important to identify which tumors will go on to develop metastasis. No prognostic biomarkers of primary breast cancer have been validated for determining patients' risk of regional nodal metastasis. Consequently, it is important to identify prognostic factors in the assessment and management of patients with primary breast cancer. Investigation of potential biomarkers that relate to breast cancer cells to escape host immune surveillance may also identify targets for preventive therapies.

### SUMMARY OF THE INVENTION

B7H3 is a ligand member of the immunoregulatory family of proteins on immune cells. In one embodiment, a method for diagnosing the progression of cancer with a high propensity of primary tumor metastasis to the lymph node or distant site is provided. Such a method may comprise obtaining a cancer tissue sample from a cancer patient, determining an expression level of B7-H3 present in the tissue sample, and diagnosing the progression of the cancer having a high propensity of primary tumor metastasis to the lymph node or distant site based upon the expression level, wherein an increased expression level correlates with an increased probability of having regional lymph nodes or organ site that are positive for metastases.

In one embodiment, the cancer tissue sample is a primary or metastatic tumor tissue specimen. In another embodiment, the cancer tissue sample is a blood specimen from a cancer patient. In some embodiments, the expression level of B7-H3 may be determined by immunohistochemistry (IHC), an anti-B7-H3 magnetic bead capture assay, or a direct qRT-PCR assay.

According to some embodiments, cancer may be selected from the group consisting of melanoma, breast cancer, and gastrointestinal cancers such as gastric cancer, colorectal, periampullary, pancreatic, liver cancer.

In another embodiment, a method for predicting nodal metastasis in breast cancer is provided. Such a method may comprise obtaining a primary breast cancer tumor tissue sample from a breast cancer patient, determining an expression level of B7-H3 present in the primary breast cancer tumor tissue sample, and diagnosing progression of breast cancer wherein an increase in the B7-H3 expression level correlates with an increase in the number of lymph nodes having metastases. The method may further comprise obtaining a lymph node tissue from a breast cancer patient and determining the expression level of B7-H3 present in the lymph node.

In a further embodiment, a method for predicting the progression of melanoma cancer is provided. Such a method may comprise obtaining a melanoma tumor tissue sample, determining an expression level of B7-H3 present in the melanoma tumor tissue sample, and diagnosing progression of melanoma, wherein an increase in B7-H3 expression correlates with an increase in primary tumor tissue metastasis.

These methods can be used for detection of metastasis in patients' blood, staging of patients disease, follow up during treatment, disease outcome prediction, and identifying B7H3 (+) circulating tumor cella (CTC) mRNA and DNA profiles. B7H3 detection in primary breast cancer can predict lymph node metastasis. The invention therefore provides prognostic utility for advance stages of metastatic disease spreading. It also provides primary tissue prediction of lymph node metastasis for breast cancer.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1 is a graph illustrating B7-H3 mRNA expression in melanoma tissues with AJCC stages I to IV and normal skin tissues. Horizontal bars indicate mean relative B7-H3 mRNA copies.

FIG. 2 is a series of representative pictures of melanoma tumor tissue illustrating IHC for B7-H3 protein expression in melanoma tissues. A, primary tumor with negative immunoreactions. B, liver metastatic tumor with strong immunoreactions. Scale bars indicate 100 μm in length. Magnification: 400×.

FIG. 3 is a series of representative pictures of immunofluorescent staining of B7-H3 expression in a melanoma cell line. A, Brightfield. B, anti-B7-H3 mAb Phycoerythrin (PE) staining. C, DAPI (4′,6-diamidino-2-phenylindole) staining. D, Combination of PE and DAPI staining. Scale bars indicate 100 μm in length. Magnification: 400×.

FIG. 4 is a series of Flow cytometric histograms analyzing B7-H3 expression (open histogram) versus antibody isotype controls (shaded histogram) in melanoma cell lines (M-1, M-101, M-111, M-12, M-14, JK0346 and MeI-B) and normal PBCs.

FIG. 5 is a graph illustrating B7-H3 mRNA expression in breast cancer and normal breast tissue. The cutoff value (dotted line) was set at 5.92×10⁻³. Horizontal bars indicate mean relative mRNA copy number.

FIG. 6 is a series of representative IHC for B7-H3 protein expression in breast cancer and normal breast tissue. A, Normal breast epithelium. Normal breast epithelial cells are not stained or very weakly stained by anti-B7-H3 mAb. B, Breast cancer cells with weak staining. C, Breast cancer cells with moderate staining. D, Breast cancer cells with strong staining. B7-H3 protein expression was detected in both the cell membrane and cytoplasm. Scale bars indicate 50 μm in length. Magnification, 400×.

FIG. 7 is a receiver operating characteristic curve (ROC) for the prediction of lymph nodes with metastasis using combined lymphovascular invasion with B7-H3 expression. The AUC was 0.732.

FIG. 8 is a graph illustrating B7-H3 mRNA expression in lymph nodes with tumor metastasis and normal lymph nodes. Horizontal bars indicate mean mRNA copy number.

FIG. 9 is a series of representative IHC for B7-H3 protein expression in lymph nodes with tumor metastasis. A, Lymph node with metastasis; Magnification, 200×. B, Lymph node with metastasis; Magnification, 400×. Scale bars indicate 100 μm in length.

FIG. 10 is a graph illustrating B7-H3 mRNA expression in various tumor cell lines.

FIG. 11 is a series of Flow cytometric histograms analyzing B7-H3 expression (open histogram) versus antibody isotype controls (shaded histogram) in osteosarcoma cell lines (U-2-OS, SJSA-1, Saos-2, MG-63 and KHOS/NP) and normal PBCs.

### DETAILED DESCRIPTION OF THE INVENTION

Methods for diagnosing the progression and early lymph node metastasis of cancer using B7-H3 as a biomarker are provided herein. In some embodiments, B7-H3 may be used as a biomarker in cancers having a high propensity of primary lymph node metastasis including, but not limited to, melanoma, breast cancer, colon cancer and gastric cancer. Metastasis is the spread of tumor cells form the site of the primary tumor to distant organ environments, and is the leading cause of death from cancer. Metastasis involving the vascular system has been well established, but in many tumors, metastasis via the lymphatic system occurs before metastasis via the vascular system, resulting in a primary lymph node metastasis prior to metastasis in distant organs. Thus, in certain types of cancers, tumor cell metastasis to regional lymph nodes often marks the first step in tumor cell progression. Examples of cancers that exhibit early lymph node metastasis include: 1) Breast cancer, wherein early stages of breast cancer metastasis frequently occurs to the regional tumor draining-lymph nodes first, followed by spread into the vascular system in more advanced disease stages. (Olson & McCall 2008; Turner et al. 2008); 2) Melanoma; and 3) Gastrointestinal cancers such as gastric cancer, colorectal cancer, pancreatic cancer, liver cancer. As an example, tumors in the large intenstine metastasize almost exclusively through the lymphatic system (Bernadette & Bielenberg 2007).

According to some embodiments, levels of B7-H3 expression in a primary tumor or in circulating tumor cells can be used to predict and/or diagnose the extent of primary nodal metastasis. Such prediction can be used to determine how far the cancer has progressed and predict the clinical outcome of the cancer.

Predicting the metastatic potential of a patient's tumor is challenging. Evidence of tumor cells in one or more lymph node is one of the first indicators of the spread of cancer. Further, lymph node metastasis is correlated with an increase risk of distant metastasis and a poor clinical outcome. Tumor cells invade the closest, or draining lymph node first (called the “sentinel node”), followed by the next node in line with the drainage flow, and so on. Sentinel lymph node (SLN) biopsy or lymphadenectomy is performed in many cancers to determine the existence and/or extent of primary nodal metastasis.

In one embodiment, prediction and/or diagnosing the progression of a cancer with a high propensity of primary lymph node metastasis is accomplished by first obtaining a cancer tissue sample from a cancer patient. In one aspect, the cancer tissue sample may be from a primary or metastatic tumor tissue specimen obtained from a cancer patient undergoing surgery or a biopsy. In another aspect, the cancer tissue sample may be a blood specimen. Blood is a type of connective tissue, and may also be considered a cancer tissue when detecting whether circulating tumor cells are present in the blood. Taking a blood sample is much less invasive than a biopsy of lymph nodes or other tumor tissues. Therefore, measurement of B7-H3 in a blood specimen would be an advantageous method for determining the existence and/or extent of primary nodal metastasis as compared to SLN biopsy. In another aspect, the cancer tissue sample may be any bodily fluid that may contain circulating tumor cells, including, but not limited to, cerebrospinal fluid (CSF), spinal fluid, synovial fluid, ascetic fluid, pericardial fluid, peritoneal fluid, or any other appropriate interstitial fluid.

As illustrated by the examples below, B7-H3 is an accurate biomarker for the detection and prediction of the progression of cancer. Therefore, according to embodiments of the disclosure, once a cancer tissue sample has been obtained, the level of B7-H3 expression may be determined. When the cancer tissue sample is a primary or metastatic tumor tissue specimen, this determination may be made by any suitable assay, including, but not limited to, immunohistochemistry or a direct qRT-PCR assay, both of which are described in detail in the examples below. When the cancer tissue sample is a blood specimen, the determination of B7-H3 expression may be made by any suitable assay, including, but not limited to, immunocytochemistry, a direct qRT-PCR assay, or an anti-B7-H3 magnetic bead capture assay, all of which are described in detail in the examples below.

Once the level of B7-H3 expression is determined, the expression level is then analyzed for diagnosing the progression of the cancer. In particular, according to one embodiment, an increased B7-H3 expression level correlates with an increased probability of having regional lymph nodes that are positive for metastases.

In another embodiment, a sample of blood or bodily fluid (e.g., cerebrospinal fluid (CSF), spinal fluid, synovial fluid, ascetic fluid, pericardial fluid, peritoneal fluid, or any other appropriate interstitial fluid) may be obtained, then a direct qRT-PCR assay is performed to determine the level of B7-H3 that is expressed in the total sample. The B7-H3 expression level is then analyzed to detect CTC in the sample of blood of bodily fluid. In particular, according to one embodiment, an increased B7-H3 expression level correlates with an increased number of CTC detected.

According to embodiments of the disclosure, methods for treating a cancer with a high propensity of primary tumor metastasis to the lymph node or distant site are provided herein. In one embodiment, detection of B7-H3(+) circulating tumor cells may be accomplished as described above. Once B7-H3(+) circulating tumor cells (CTC) are detected, treatment may comprise administering a therapeutically effective amount of a pharmaceutical composition. In some embodiments, the pharmaceutical composition may include a B7-H3 ligand or a B7-H3 binding molecule to target the B7-H3 that is present on the CTC, conjugated to a cytotoxic drug to kill the CTC.

In some embodiments, the B7-H3 ligand or binding molecule may be an antibody or functional fragment thereof. An antibody or functional fragment thereof refers to an immunoglobulin molecule that specifically binds to, or is immunologically reactive with a particular antigen, and includes both polyclonal and monoclonal antibodies. The term also includes genetically engineered or otherwise modified forms of immunoglobulins, such as chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′).sub.2, Fab, Fv, rIgG, and scFv fragments. The term scFv refers to a single chain Fv antibody in which the variable domains of the heavy chain and of the light chain of a traditional two chain antibody have been joined to form one chain. In another embodiment, the B7-H3 ligand may be a protein peptide, fusion protein, peptibody, chimeric protein, small molecule, or other biologic.

In some embodiments, the cytotoxic drug may be a chemotherapeutic or other suitable agent, including alkylating agents, antimetabolites, anthracyclines, plant alkaloids, topoisomerase inhibitors, and other antitumour agents. All of these drugs affect cell division or DNA synthesis and function in some way. In other embodiments, the cytotoxic drug may be a targeted therapy, which include agents that do not directly interfere with DNA. These include monoclonal antibodies and tyrosine kinase inhibitors which directly target a molecular abnormality in certain types of cancer.

The pharmaceutical compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W [1995] Easton Pa., Mack Publishing Company, 19th ed.) describes formulations that can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The pharmaceutical composition described above is administered and dosed in accordance with good medical practice, taking into account the clinical condition of the individual patient, the site and method of administration, scheduling of administration, patient age, sex, body weight, and other factors known to medical practitioners. The therapeutically effective amount for purposes herein is thus determined by such considerations as are known in the art. For example, an effect amount of the pharmaceutical composition is that amount necessary to reduce the number of circulating cytokines to prevent metastasis. The amount of the pharmaceutical composition must be effective to achieve improvement including but not limited to total prevention and to improved survival rate or more rapid recovery, or improvement or elimination of symptoms associated with the metastatic cancer being treated and other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the patient and the route of administration.

### Melanoma Studies

In one embodiment, the methods described herein are directed to predicting the progression of melanoma cancer. The presence of CTC in melanoma patients has been shown to indicate aggressive disease and poor prognosis (Goto et al. 2008; Koyanagi et al. 2005; Mocellin et al. 2005; Takeuchi et al. 2003).

The studies described in the examples below demonstrate the expression of B7-H3 ligand by melanoma cells and the relation to melanoma progression. The expression of B7-H3 on melanomas was shown to be found in more aggressive melanomas particularly metastatic tumors. The presence of B7-H3(+) CTC found in metastatic melanoma patient blood would suggest a potential diagnostic biomarker for the spread of systemic disease. B7-H3(+) melanoma cells may be a potential target for therapy and also a diagnostic biomarker for melanoma progression.

The studies below are the first demonstrating the characterization of B7-H3 expression of human melanomas relating to metastasis. B7-H3 expression was demonstrated by melanoma cells in melanoma cell lines and PEAT specimens by using qRT-PCR, flow cytometry and IHC. In addition to these methods, an immunobead mAb capture assay may be used to detect the presence of B7-H3(+) circulating tumor cells in the blood. In blood specimens, B7-H3 protein expression on the cell surface of several melanoma cell lines cells was verified using immunofluorescence staining and flow cytometric analysis.

The B7-H3 molecule was first cloned from a human dendritic cell-derived cDNA library in 2001 (Chapoval et al. 2001). At present, its receptor is not known in humans. Although B7-H3 is suggested to provide both costimulatory or coinhibitory signals to regulate T-cell-mediated immune responses, however, more compelling evidence of the latter function seems to be predominant. Investigators have reported that B7-H3 molecule acts as a coinhibitory regulator of antitumor immunity in neuroblastoma, non-small cell lung cancer, and prostate cancer (Castriconi et al. 2004; Roth et al. 2007; Sun et al. 2006). Recently, in prostate cancer, B7-H3 expression was shown as an independent predictor of tumor progression (Roth et al. 2007).

The studies below demonstrate that the B7-H3 expression level was significantly higher in metastatic melanomas compared to primary melanomas. These results reflect a relationship between B7-H3 expression and melanoma progression. B7-H3 expression may facilitate a mechanism of melanoma cell escape from host immune responses.

The clinical utility of CTC detection by a multimarker qRT-PCR assay of blood from patients undergoing treatment of metastatic melanoma has been reported (Koyanagi et al. 2005a; Koyanagi et al. 2006; Hoon et al. 2000; Hoon et al. 1995; Koyanagi et al. 2005b). As described in the examples below, a four marker assay for melanoma cells may be performed to confirm the CTC isolation by the anti-B7-H3 magnetic bead capture assay, or to further characterize the CTC. The four marker assay may use the following melanoma markers: melanoma antigen recognized by T-cells-1 (MART-1), melanoma antigen gene-A3 family (MAGE-A3), high molecular weight-melanoma-associated antigen (HMW-MAA), and tyrosinase-related protein-2 (TRP-2). The efficacy of the four marker assay depends on efficient isolation of CTC from peripheral blood, which in turn requires a cell-surface marker that is frequently highly expressed in metastatic melanoma cells and not in normal blood cells. B7-H3 expression on CTCs provides for efficient immunoselection of B7-H3(+) CTCs from blood.

Immunomagnetic bead detection is useful for the isolation of tumor cells from blood and bone marrow with several types of malignancies (Bruland et al. 2005; Faye et al. 2004; Flatmark et al. 2002; Kielhorn et al. 2002; Werther et al. 2000). This approach may be used for efficient isolation of B7-H3(+) CTC from the blood of metastatic melanoma patients. A high frequency of B7-H3(+) CTC in blood of advanced stage melanoma patients would strongly suggest that B7-H3 as an aggressive phenotype biomarker of cutaneous melanoma progression and that CTC are present in metastatic melanoma patients and more frequent in advancing stages of tumor progression.

Future studies on the functional role of B7-H3 expression in melanoma patients may allow the development of new targeted therapies directed to the B7-H3 signaling pathway. Identification of the human B7-H3 receptor is also important for the development of future therapeutics. At present, clinical trials of anti-CTLA-4 monoclonal antibody therapy are being carried out in melanoma patients to reverse immune suppression and promote anti-melanoma immune responses (O'Day et al. 2007). Recently, clinical studies blocking CTLA-4 using anti-CTLA-4 human monoclonal antibodies (Tremelimumab and Ipilimumab) have shown encouraging clinical responses. Both have shown some clinical success in treatment of advanced-stage melanoma patients (Beck et al. 2006; Hodi et al. 2003; O'Mahoney et al. 2007; Ribas et al. 2005; Small et al. 2007).

In summary, the data set forth herein shows that B7-H3 expression on primary and metastatic tumors significantly correlate with progression of melanoma. An immunomagnetic bead isolation of B7-H3(+) CTC can demonstrate the presence of melanoma B7-H3(+) cells in the blood of patients with metastatic disease. Furthermore, CTC in the blood or body fluids (e.g., cerebrospinal fluid (CSF), spinal fluid, synovial fluid, ascetic fluid, pericardial fluid, peritoneal fluid, or any other appropriate interstitial fluid) can be detected by a direct qRT-PCR assay, requiring an RNA extraction step, but no separate CTC isolation step (Koyanagi et al. 2005b). Therefore, B7-H3 is a clinically useful molecular biomarker for melanoma progression and detecting CTC in melanoma patients. B7-H3 ligand may also be a potential immunotherapeutic target in melanoma patients.

### Breast Cancer

In another embodiment, the methods described herein are directed to predicting primary nodal metastasis in breast cancer. The study described in the examples below is the first to demonstrate B7-H3 expression in human breast cancer tissues from primary tumors and regional nodal metastasis. It was demonstrated that B7-H3 expression of primary tumor is a significant factor in predicting nodal metastasis in a multivariate analysis. To date, there have not been any validated prognostic factors in primary breast cancer that can identify metastasis to regional tumor-draining lymph nodes. B7-H3 expression was correlated with a primary tumor's size and LVI, with AJCC stage of breast cancer, and with the presence and extent of metastasis in axillary SLN and NSLN nodes. Interestingly, B7-H3 protein was expressed in all primary tumor-draining lymph nodes that contained metastasis.

Although it is thought that B7-H3 may be involved in downregulation and escape of host immunity, its clinical and functional significance is still unclear. B7-H3 co-inhibitory regulatory immune activities have been well documented in both mouse and human studies (Chapoval et al. 2001; Flies & Chen 2007; Greenwald et al. 2005; Zang & Allison 2007; Chen 2004). B7 ligand family members have been found on various tumor cells and suggested as a facilitator of immune escape by neutralizing host immunity (Castriconi et al. 2004; Roth et al. 2007; Sun et al. 2006; Thompson et al. 2004; Tirapu et al. 2006). In vivo tissue studies suggested that B7-H3 was involved in inhibitory immunoregulation in non-small cell lung and prostate cancers (Roth et al. 2007; Sun et al. 2006). These studies indicate that B7-H3 coregulatory molecules expressed by tumor cells play a role in suppressing host anti-tumor immunity including Natural Killer (NK) cell activity (Castriconi et al. 2004). According to the study below, the co-regulatory molecule B7-H3 might function in a similar manner in breast cancer, that is, B7-H3 expression by primary tumors and nodal metastases may promote the progression of breast cancer by suppressing T-cell anti-tumor immunity.

While several investigators have reported that B7-H3 is a negative regulator of T-cell function (Suh et al. 2003; Ling et al. 2003; Prasad et al. 2004), there is no clear consensus about the functional significance of B7-H3 expression by tumor cells. Some studies suggest a link between B7-H3 expression and progression of tumor malignancy, and some have suggested a relation between B7-H3 expression and clinicopathological characteristics, including prognosis, in some malignancies (Castriconi et al. 2004; Roth et al. 2007; Sun et al. 2006). Other groups have also suggested B7-H3 as a factor related to lymph node metastasis in other cancers (Castriconi et al. 2004; Crispen et al. 2008). In prostate cancer, patients with marked expression of B7-H3 had a worse prognosis compared to patients with weak B7-H3 expression (Roth et al. 2007).

As demonstrated below, B7-H3 is strongly expressed in breast cancer cells and B7-H3 expression is related to progression of primary breast cancer to axillary lymph nodes. Thus, B7-H3 expression in preoperative biopsy specimens may be used as a predictor of lymph node metastasis. Further identification and understanding of the B7-H3 signaling pathway and potential receptor(s) may offer new therapeutic strategies in primary breast cancer. Colonization of cancer cells has been previously shown in immunosuppressed draining lymph nodes by Hoon et al. (Hoon et al. 1987a; Hoon et al. 1987b). The presence of B7-H3 on regional node metastases of breast cancer suggests that B7-H3 may allow cancer cells to escape immune-based surveillance resulting in such colonization. To date, studies on immune escape of micrometastasis in lymph nodes have focused on the immune cells. Metastatic tumor cells bearing cell-surface immunoregulatory molecules may also significantly contribute to escape from immune effector cells. B7-H3 has been shown to suppress type I T-helper cell responses and regulate cytokine activity (Zang & Allison 2007; Suh et al. 2003). Therefore, tumor cells spreading regionally and systemically that express B7-H3 may have a significant survival advantage. Interestingly B7-H3 expression appears to be a regulatory factor that the tumor cells have utilized the immune system to escape immune surveillance or induce immune tolerance. B7-H3 ligand appears to be the only member of the B7-H ligand family to date that is significantly expressed by human tumor cells and relates to tumor progression.

The data set forth herein reflects that B7-H3 expression by breast cancer cells is a tumor progression factor that is a predictor of early regional nodal metastasis. The expression of B7H3 by primary breast cancer is therefore an important predictor of aggressive regional breast cancer progression and can also be a potential therapeutic target.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### EXAMPLES

### Example 1

### B7-H3 Expression in Melanoma Tumor Specimens is a Tumor Progression Factor

The purpose of this study was to assess B7-H3 expression in primary and metastatic melanomas to determine its relation to disease progression.

Materials and Methods

Tissues. Paraffin-embedded archival tissue (PEAT) specimens were obtained for primary tumors from 57 patients with AJCC stage I (n=22), stage II (n=14), and stage III (n=21) melanoma. PEAT specimens also were obtained for metastatic tumors from 43 patients with AJCC stage III (n=23) and IV (n=20) melanoma. All patients underwent surgical resection at Saint John's Health Center (SJHC, Santa Monica, Calif.) from 1997 through 2006. Thirteen PEAT specimens of normal skin were used as controls. These skin PEAT specimens were histopathologically confirmed to be tumor free by a surgical pathologist.

Immunohistochemistry. Five μm-thick PEAT sections were cut and incubated on glass slides at 50° C. overnight for IHC. These PEAT sections were deparaffinized with xylene, rehydrated with a graded series of ethanol, and autoclaved in EDTA buffer (1 mM, pH 8.0) at 121° C. for 15 min to retrieve the antigen. After cooling at room temperature, peroxidase blocking reagent (DakoCytomation, Carpinteria, Calif.) was used to block the endogenous peroxidase for 5 min. Non-specific binding was blocked at room temperature for 5 min with protein block serum-free (DakoCytomation). The tissue sections were incubated at room temperature for 1 hr with anti-human B7-H3 polyclonal antibody (100 μg/ml; R&D Systems, Minneapolis, Minn.) diluted 1:10 in phosphate-buffered saline (PBS). After three 5-min washes in PBS, the reaction for B7-H3 was developed using a labeled streptavidin biotin (LSAB) method (LSAB+Kit; DakoCytomation) and visualized using VIP Substrate Kit (Vector Laboratories, Burlingame, Calif.). The negative controls consisted of sections treated with normal goat serum (Santa Cruz Biotechnology. Inc., Santa Cruz, Calif.) instead of primary antibody under the same conditions.

The IHC analysis for B7-H3 was assessed and scored by two independent investigators (T.A. and N.N.). The IHC results for B7-H3 protein expression were classified as having strong (+++), moderate (++), weak (+), or negative immunoreaction (−). B7-H3 protein expression was evaluated using light microscopy (400×).

RNA extraction. For RNA extraction of individual PEAT specimens, 10 sections of 10 μm-thick tissues were cut using a microtome and disposable sterile blade and placed in a sterile microcentrifuge tube. These sections were deparaffinized with xylene and washed with 100% ethanol as previously described (Umetani et al. 2005). In PEAT specimens from 43 metastatic melanoma sites, 10 sections of 10 μm-thick tissues were cut. After deparaffinization, the sections were stained with hematoxylin, and tumor cells were accurately microdissected under a microscope as previously described (Hoon et al. 2000; Hoon et al. 1995). All sections were incubated by a proteinase K digestion buffer (Ambion, Austin, Tex.) at 50° C. for 3 hrs as previously described (Umetani et al. 2005). Total RNA from PEAT specimens were extracted, isolated, and purified using a modified RNAWiz (Ambion) phenol-chloroform extraction method as previously described (Umetani et al. 2005). Pellet Paint NF (EMD Biosciences. Inc., San Diego) was used as a carrier for precipitation. The concentration, purity, and amount of total RNA were measured by ultraviolet spectrophotometry and RIBOGreen detection assay (Molecular Probes, Invitrogen) as previously described (Rosenberg et al. 1990; Umetani et al. 2005).

Primers and probes. Primer and probe sequences of B7-H3 were designed to assess B7-H3 mRNA expression in PEAT specimens of melanoma patients. The forward primer, fluorescence resonance energy transfer probe sequence, and reverse primer for B7-H3 were as follows:

(forward)
(SEQ ID NO: 1)
5′-GACAGCAAAGAAGATGATGGA-3′
(probe)
(SEQ ID NO: 2)
5′-FAM-CCTCCCTACAGCTCCTACCCTCTGG-BHQ-1-3′
(reverse)
(SEQ ID NO: 3)
5′-ACCTGTCAGAGCAGGATGC-3′

In addition, the glyceraldehyde-3-phosphate dehydrogenase (GAPDH), housekeeping gene was used as an internal control to confirm RNA integrity. The GAPDH probe is as follows:

(SEQ ID NO: 4)
5′-FAM-CAGCAATGCCTCCTGCACCACCAA-BHQ-1-3′.

qRT-PCR assay. All reverse transcription reactions of total RNA were done using Moloney murine leukemia virus reverse transcriptase (Promega, Madison, Wis.), oligo-dT primer (Gene Link, Hawthorne, N.Y.) and random hexamers (Roche Diagnostics, Indianapolis, Ind.) as previously described (Takeuchi et al. 2003; Rosenberg et al. 1990). The qRT-PCR assay was performed with the iCycler iQ Real-Time Thermocycler Detection System (Bio-Rad Laboratories, Hercules, Calif.) as previously described (Takeuchi et al. 2003; Rosenberg et al. 1990). For each reaction, complementary DNA from a total of 250 ng of RNA was used with a reaction mixture containing each primer, probe, and iTaq custom Supermix (Bio-Rad).

Samples were amplified with a precycling hold at 95° C. for 10 min, followed by optimized cycles of denaturation for each marker at 95° C. for 60 sec (40 cycles for both GAPDH and B7-H3), annealing for 60 sec (at 63° C. for B7-H3 and 55° C. for GAPDH), and extension at 72° C. for 60 sec. Specific plasmids for external controls of each marker were synthesized as described previously (Imai et al. 1982). Standard curves for each assay were generated using a threshold cycle of six serial dilutions of plasmid templates (10⁶-10¹ copies), and the mRNA copy number was calculated using the iCycler iQ Real-Time Thermocycler Detection System Software (Bio-Rad). Each assay was repeated in duplicate with positive (ME-2 cells) and reagent controls (reagent alone) for verification of the qRT-PCR assay.

Statistical analysis. The Wilcoxon rank sum test was used to assess the difference in B7-H3 mRNA copy numbers between melanoma tissues with each AJCC stage and normal skin in PEAT specimens. Kruskal-Wallis test was used to identify AJCC stage-related differences in B7-H3 mRNA copy numbers. All statistical calculations were performed using SAS statistical software (SAS Institute. Inc., Cary, N.C.). A P value of <0.05 was considered statistically significant.

B7-H3 mRNA Expression in Melanoma Tissue Specimens.

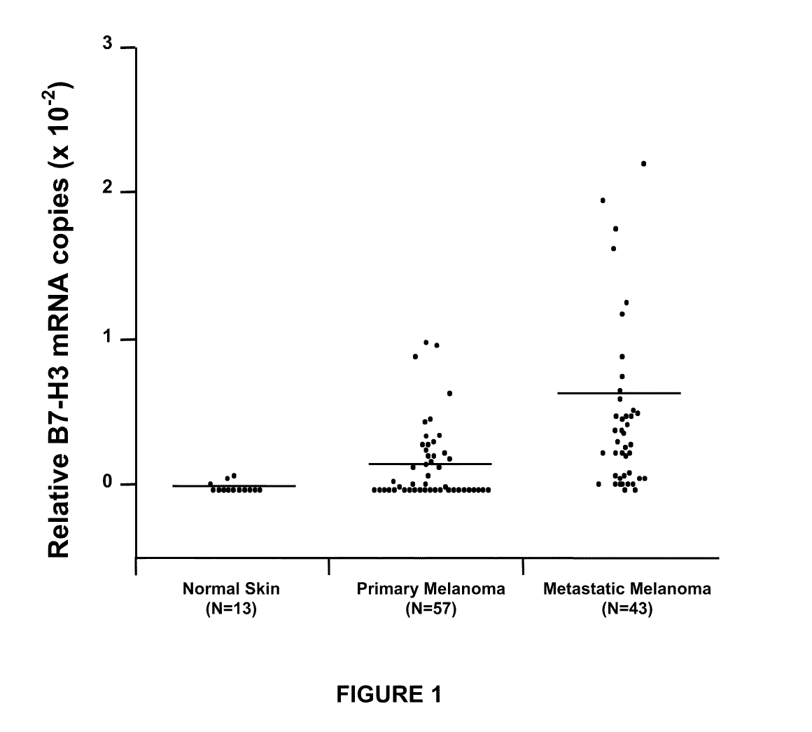

To assess B7-H3 mRNA expression in melanoma tissues, a qRT-PCR assay for B7-H3 mRNA was performed on PEAT specimens of primary and metastatic melanoma. Thirteen PEAT specimens of normal skin were used as controls. Mean (±SD) relative B7-H3 mRNA copies in primary tumors from patients with AJCC stages I, II, III melanoma were 7.67×10⁻⁴±1.29×10⁻³ (range, 0−3.6×10⁻³), 2.28×10⁻³±3.12×10⁻³ (range, 0−9.93×10⁻³), 1.71×10⁻³±2.86×10⁻³ (range, 0-1.0×10⁻²), respectively. For AJCC stage III and IV metastatic tumors, B7-H3 mRNA copies were 4.76×10⁻³±6.23×10⁻³ (range, 0−2.24×10⁻²), and 5.10×10⁻³±4.74×10⁻³ (range, 0−1.79×10⁻²), respectively. B7-H3 mRNA copy number distribution of normal, primary melanomas, and metastatic melanomas are shown in FIG. 1. B7-H3 mRNA copy numbers were significantly higher in primary melanoma tissues with AJCC stages I, II, and III, and metastatic melanoma tissues with AJCC stage III and IV than compared to normal skins (P=0.036, P=0.039, P=0.0001, and P<0.0001, respectively). AJCC stage-related tumor differences in B7-H3 mRNA copy numbers were determined highly significant relative to advancing disease (P<0.0001).

B7-H3 Protein Expression by Melanoma Tissue Specimens.

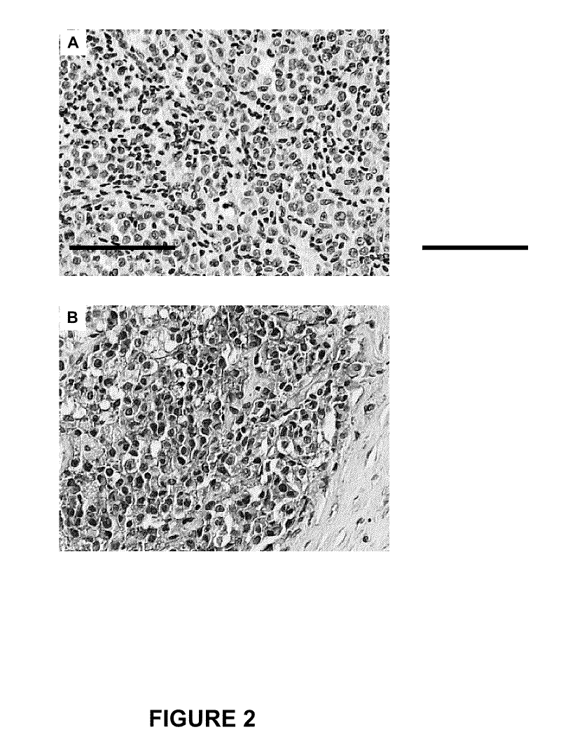

The presence of B7-H3 protein expression in melanoma tissue was determined by IHC. B7-H3 protein expression demonstrated 6 of the 57 primary melanomas and 20 of the 43 metastatic melanomas were positive by IHC. B7-H3 expression was identified in the cell membrane and/or cytoplasm. Although tumors had varied intensities for B7-H3 staining, moderate or strong immunoreaction was detected in 3 of 6 (50%) primary melanomas and 16 of 20 (80%) metastatic melanomas (FIG. 2A, B). IHC analysis demonstrated that B7-H3 expression was higher in metastatic tumors than primary tumors.

### Example 2

### B7-H3 is an Efficient Cell Surface Marker for Detecting Metastatic Melanoma Cells

Materials and Methods

Melanoma cell lines. Seven established human melanoma cell lines (M-1, M-101, M-111, M-12, M-14, JK0346 and MeI-B) were used in this study. These melanoma lines were cultured in GIBCO RPMI 1640 (Invitrogen, Carlsbad, Calif.), and supplemented with 10% heat-inactivated fetal bovine serum (FBS), 100 units/mL penicillin, and 100 units/mL streptomycin as previously described (Goto et al. 2008). All cell lines were grown at 37° C. in a humidified atmosphere at 5% CO₂ as previously described (Nakagawa et al. 2007). Melanocyte primary cultures were commercially (Lonza Inc., Allendale, N.J.) obtained and grown in specific tissue culture medium as suggested by the manufacturers.

Flow cytometry. Flow cytometric analysis was performed using the BD FACSCalibur System (BD Biosciences, San Jose, Calif.). After washing in flow cytometry buffer (PBS with 1% FBS) to block nonspecific binding, 1×10⁶ melanoma cells were incubated at 4° C. for 1 hr with 1 μg of anti-B7-H3 mAb (R& D Systems, Minneapolis, Minn.). These cells were stained at 4° C. for 30 min with an optimal amount of phycoerythrin (PE)-labeled F(ab′)₂ fragment of goat anti-mouse IgG (Santa Cruz Biotechnology) after washing in flow cytometry buffer. Cells were fixed in 4% formaldehyde and analyzed using Cell Quest software (Becton Dickinson, Franklin Lakes, N.J.). Isotype-matched antibodies were used as negative controls.

Immunocytochemistry. Melanoma cells were cultured on Lab-Tek II chamber slides (Nalge Nunc International Corp., Naperville, Ill.) and fixed with 4% paraformaldehyde in PBS for 10 min after washing in PBS. Melanoma cells were stained using anti-B7-H3 mAb (R& D Systems, Minneapolis, Minn.) and PE-conjugated goat anti-mouse secondary antibody (Santa Cruz Biotechnology) at room temperature for 1 hr. Slides were mounted with Vectashield Mounting Medium containing 4′,6-diamidino-2-phenylindole (DAPI) for nuclear staining (Vector Laboratories, Burlingame, Calif.). Cells were analyzed using a Nikon Eclipse Ti fluorescence microscope (Nikon Instruments Inc, Melville, N.Y.).

Immunofluorescent Staining and Flow Cytometric Analysis of B7-H3 Expression.

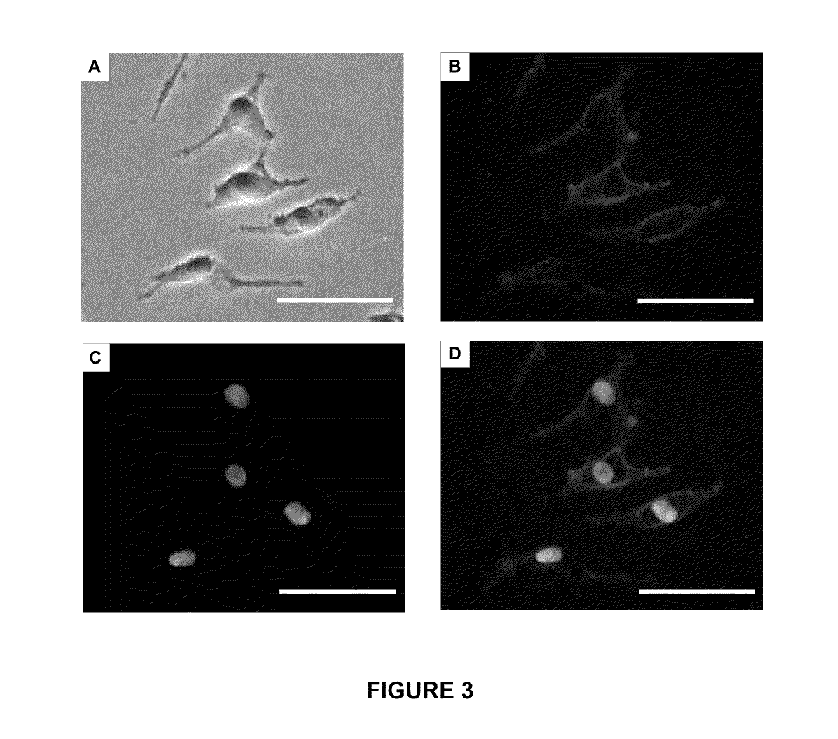

Immunofluorescent staining of melanoma cell lines demonstrated strong B7-H3 protein expression on the cell surface (FIG. 3A-D). B7-H3 immunofluorescent analysis of primary melanocytes from culture under optimal conditions demonstrated no significant immunostaining (data not shown).

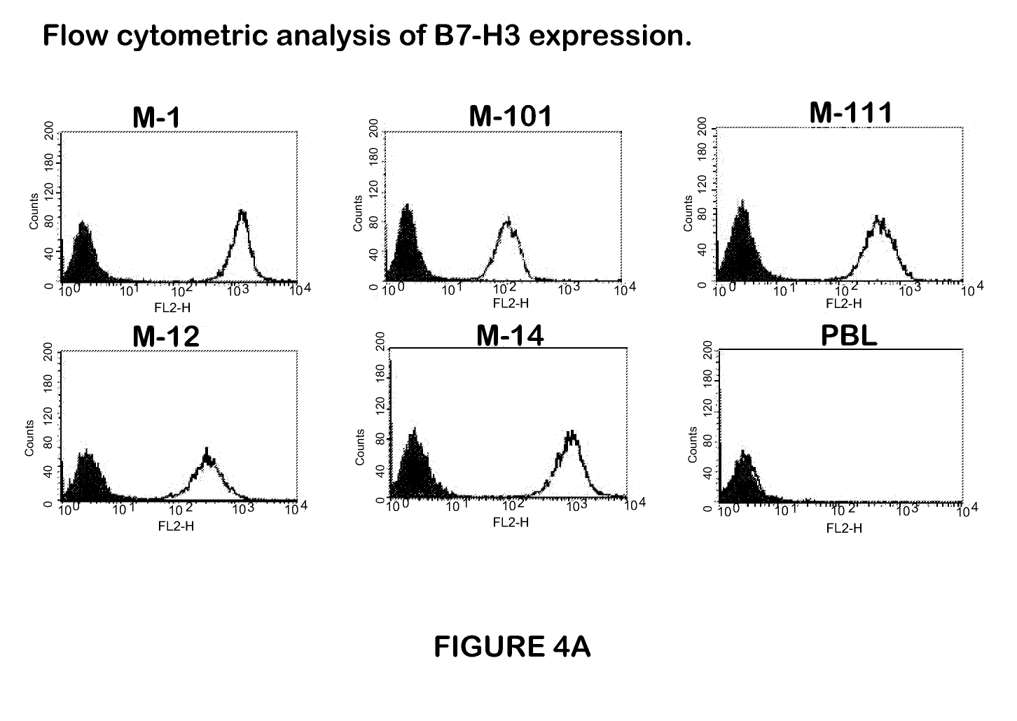

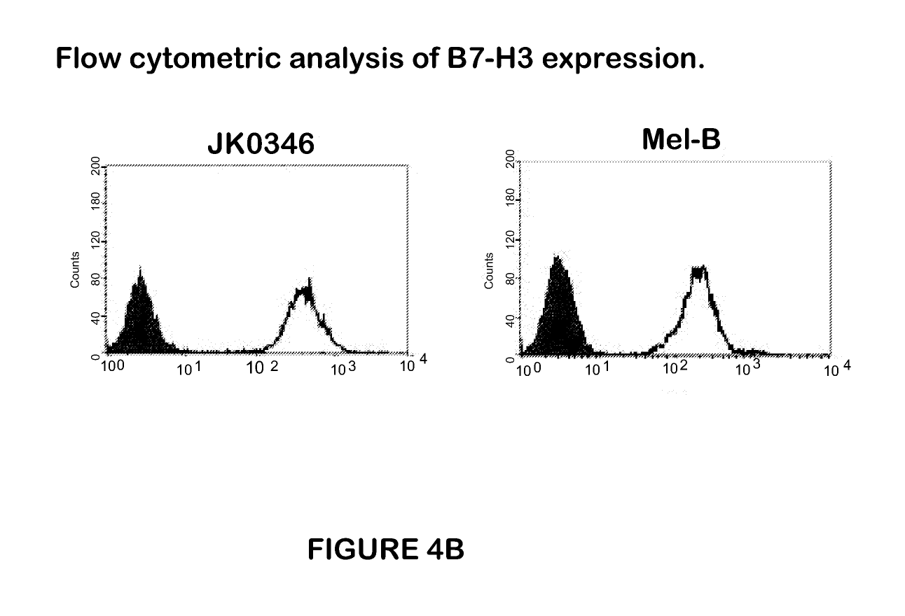

Melanoma lines M-1, M-101, M-111, M-12, M-14, JK0346 and MeI-B, along with PBCs from two healthy volunteers as negative controls, were assessed to determine whether B7-H3 is a good marker for determining the presence of metastatic melanoma cells. Flow cytometric analysis demonstrated that B7-H3 was highly expressed on the cell surface of all melanoma cell lines (n=5) and not on normal donor PBCs as shown in FIG. 4 (B7-H3 expression shown by open histogram).

### Example 3

### B7-H3 Expression in Breast Cancer Tumor Specimens is a Progression Factor for Early Stages of Primary Breast Tumor

The purpose of this study was to investigate B7-H3 expression in primary breast tumors and its association with progression to develop regional nodal metastasis.

Materials and Methods

Breast cancer cell lines. Six established breast cancer cell lines (MCF7, T-47D, ZR-75-1, OR-090-1, 734/B, and BT-20) were cultured in GIBCO RPMI 1640 (Invitrogen, Carlsbad, Calif.) supplemented with 10% heat-inactivated fetal bovine serum (FBS), 100 units/mL penicillin and 100 units/mL streptomycin. All cell lines were grown at 37° C. in a humidified atmosphere containing 5% CO₂, as previously described (Nakagawa et al. 2007).

Patient Specimens. All specimens evaluated were from AJCC stage I-III breast cancer patients treated at John Wayne Cancer Institute (JWCI) at Saint John's Health Center (SJHC), Santa Monica, Calif. between 1997 and 2002. The use of specimens, clinical information, and human subject consent were approved by the SJHC/JWCI Institutional Review Board for specimens from all patients.

Primary tumor specimens from 82 patients with invasive breast cancer who underwent resection of the primary tumor plus sentinel lymph node (SLN) and/or axillary lymph node (ALN) dissection were assessed. Of these, 66 patients underwent SLN dissection (SLND). Patients with carcinoma in situ were excluded from this study. Tumors were classified and staged according to the American Joint Committee on Cancer (AJCC) (Singletary & Connolly 2006). In addition, control tumors and lymph nodes were collected for the studies. Seventeen disease-free patients' breast normal paraffin-embedded archival tissue (PEAT) specimens were used as negative controls. Furthermore, 24 SLNs with H&E defined metastasis were collected and 10 tumor-free SLN from different patients were used as positive and negative controls, respectively. These PEAT specimens were histopathologically evaluated by a surgical pathologist. All laboratory research investigators were blinded as to patient disease status.

RNA extraction. Tri-Reagent (Molecular Research Center, Inc., Cincinnati, Ohio) was used to extract total RNA from cell lines and blood specimens as previously described (Takeuchi et al. 2004; Koyanagi et al. 2006). For RNA extraction from each PEAT specimen, 10 sections of tumor or normal/benign fibrocystic disease tissue, each 10 μm in thickness, were cut using a microtome and disposable sterile blade and placed in a 2-ml sterile microcentrifuge tube. These sections were incubated in proteinase K (Ambion, Austin, Tex.) at 50° C. for 3 hrs after deparaffinization with xylene and three washings with 100% ethanol, as previously described (Goto et al. 2006). Total RNA from PEAT specimens was extracted, isolated, and purified using a modified RNAWiz (Ambion) phenol-chloroform extraction method, as previously described (Goto et al. 2006). Pellet Paint NF (EMD Biosciences, Inc., San Diego, Calif.) was used as a carrier for precipitation. The concentration of total RNA integrity was determined by using ultraviolet spectrophotometry and RIBOGreen detection assay (Invitrogen) as previously described (Goto et al. 2006).

Primers and probes. Primer and probe sequences for a quantitative real-time reverse transcription-polymerase chain reaction (qRT) assay were designed and verified using Human BLAST Search, Primer3, and NCBI BLAST software, as previously described (Kim et al. 2005). To avoid the amplification and detection of contaminating genomic DNA, primer and probe sequences were designed to amplify at least one exon-exon junction. The primers and fluorescence resonance energy transfer probe sequences of B7-H3 and glyceraldehyde-3-phosphate dehydrogenase (GAPDH) as previously described (Takeuchi et al. 2004) and B7-H3, (forward) 5′-GACAGCAAAGAAGATGATGGA-3′, (probe) 5′-FAM-CCTCCCTACAGCTCCTA CCCTCTGG-BHQ-1-3′, (reverse) 5′-ACCTGTCAGAGCAGGATGC-3′. GAPDH, a housekeeping gene, was used as an internal control to confirm RNA quality, integrity, and normalize samples for analysis.

qRT assay. All reverse-transcription reactions of total RNA were done using Moloney murine leukemia virus reverse transcriptase (Promega, Madison, Wis.), oligo-dT primer (Gene Link, Hawthorne, N.Y.) and random hexamers in PEAT specimens (Roche Diagnostics, Indianapolis, Ind.), as previously described (Kim et al. 2005). The qRT assay was performed with the iCycler iQ Real-Time Thermocycler Detection System (Bio-Rad Laboratories, Hercules, Calif.) as previously described (Kim et al. 2005). For each reaction, cDNA from a total of 250 ng of RNA was used in a reaction mixture containing each primer, probe, and AccuQuant custom qPCR Supermix (Quanta Bioscience). The amplification profile consisted of a precycling hold at 95° C. for 10 min, followed by 45 cycles of denaturation at 95° C. for 60 sec, annealing for 60 sec (63° C. for B7-H3, 55° C. for GAPDH), and extension at 72° C. for 60 sec. Specific cDNA-containing plasmids for each biomarker were synthesized as described previously (Takeuchi et al. 2003) and used to construct standard curves based on the threshold cycles of six serial dilutions of plasmid templates (10⁶-10¹ copies). The mRNA copy number was calculated by the iCycler iQ Real-Time Detection System Software (Bio-Rad). Each assay was repeated in duplicate with a positive control (breast cancer cell line), negative controls and no template controls (reagent alone) for the verification of qRT assays. Based on this standard curve with six serial dilutions of plasmid templates, absolute copy numbers were calculated in qRT assays as previously described (Koyanagi et al. 2005b). B7-H3 mRNA copy numbers were normalized by GAPDH mRNA copy numbers and are presented as the relative B7-H3 mRNA copies (absolute B7-H3 mRNA copies/absolute GAPDH mRNA copies). The cutoff point was determined as the mean relative B7-H3 mRNA copy number plus 3 SD of the mean relative copy number for normal breast tissues; cutoff value was set at 5.92×10⁻³. This value was above the relative B7-H3 mRNA copies of all normal breast tissues. When the relative B7-H3 mRNA copy number of a specimen was above the cutoff value, the sample was considered positive for B7-H3 mRNA expression. For comparison to clinical/pathology parameters, B7-H3 binary values (established cutoff above normal tissue values) were used for analysis.

Immunohistochemistry. Five μm-thick PEAT sections were incubated on slides at 50° C. overnight for immunohistochemistry (IHC). These PEAT sections were deparaffinized with xylene, rehydrated with a graded series of ethanol, and heated in EDTA buffer (1 mM, pH 8.0) at 121° C. for 15 min to activate the antigen. After cooling at room temperature, endogenous peroxidase was blocked by Peroxidase Blocking Reagent (DakoCytomation, Carpinteria, Calif.) for 5 min. Non-specific binding was blocked at room temperature for 5 min with Protein Block Serum-Free (DakoCytomation). The tissue sections were incubated at room temperature for 60 min with a goat anti-human B7-H3 polyclonal antibody (Ab) (100 μg/ml; R&D Systems, Minneapolis, Minn.) diluted 1:10 in phosphate-buffered saline (PBS). After three 5-min washes in PBS, the reaction for anti-B7-H3 Ab was treated using a labeled streptavidin biotin (LSAB) method (LSAB+Kit; DakoCytomation) and developed with diaminobenzidine tetrahydrochloride. The negative controls consisted of sections treated with normal goat serum (Santa Cruz Biotechnology Inc., Santa Cruz, Calif.) instead of primary antibody under the same conditions.

Two independent investigators, who were blinded to the clinicopathological data of the patients and the results of qRT assay, evaluated the immunoreaction for B7-H3. The IHC results for B7-H3 protein expression were classified into 4 groups: strong immunoreaction (+++), moderate immunoreaction (++), weak immunoreaction (+), and negative immunoreaction (−) by each reader. The B7-H3 protein expression was evaluated in 10 fields under a Nikon Eclipse Ti (200× power) microscope.

Immunocytochemistry. Breast cancer cells were cultured on BD Falcon culture slides (BD Biosciences, Bedford, Mass.) and fixed with 4% paraformaldehyde in PBS for 10 min after washing in PBS. Cells were stained using the primary B7-H3 mAb (R& D Systems) and FITC-conjugated rabbit anti-goat secondary antibody (Santa Cruz Biotechnology) at room temperature for 1 hr. Slides were mounted with Vectashield Mounting Medium containing 4′,6-diamidino-2-phenylindole (DAPI) for nuclear staining (Vector Laboratories, Burlingame, Calif.). Cells were then analyzed using a Nikon Eclipse Ti fluorescence microscope.

Biostatistical analysis. The Wilcoxon rank sum test was used to assess differences in B7-H3 mRNA expression between primary breast cancers and normal breast tissues, and between tumor-positive and tumor-negative lymph nodes. Chi-square and Fisher's exact tests were used to compare categorical clinicopathologic factors according to negative or positive expression of B7-H3 by tissue from the breast or axillary lymph nodes (see Methods and Materials). The contribution of B7-H3 expression status and clinicopathological factors in the prediction of lymph node metastasis was evaluated by univariate and multivariate logistic regression. Stepwise procedure was used for the covariate selection. Receiver operating characteristic (ROC) curves were constructed to analyze the predictive power of B7-H3 mRNA expression for the detection of patients with lymph node metastasis. The area under the curve (AUC) was computed via numerical integration of the ROC curve. All statistical calculations were performed using SAS statistical software (SAS Institute. Inc., Cary, N.C.). A P value of <0.05 was considered statistically significant. Studies were followed by the guidelines of the reporting recommendations for tumor biomarker prognostic studies (McShane et al. 2005).

B7-H3 mRNA in Cell Lines and Primary Tumors

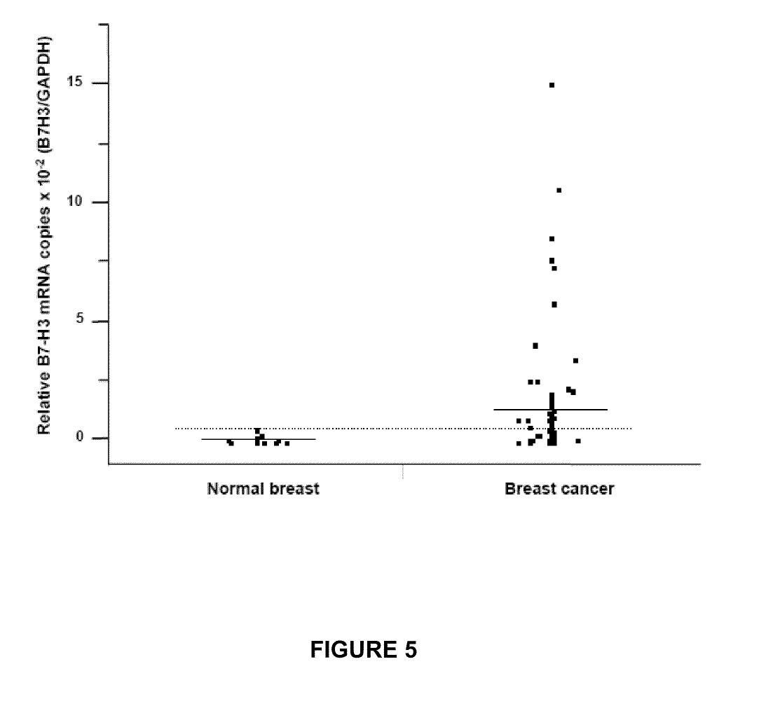

B7-H3 mRNA expression was assessed by an optimized qRT-PCR assay in six breast cancer cell lines, 82 PEAT specimens from primary breast tumors, and 17 PEAT specimens from normal/benign fibrocystic breast tissue. Breast cancer cell lines were assessed initially, showing absolute mRNA copies of B7-H3 ranging from 7.86×10⁴ to 2.53×10⁵. Mean calculated relative B7-H3 mRNA copy number (±SD) was 1.97×10⁻²±1.11×10⁻² (range, 1.19×10⁻²-3.69×10⁻²). These studies demonstrated breast cancer lines expressed B7-H3 and in variable levels. Next, primary breast tumors were assessed under the optimized qRT-PCR assay. Among the 82 primary breast tumor specimens, absolute mRNA copies of B7-H3 and GAPDH ranged from 0 to 7.43×10², and from 4.73×10¹ to 1.64×10⁴, respectively. Mean relative B7-H3 mRNA copy number (±SD) was 1.27×10⁻²±2.53×10⁻² (range, 0−1.51×10⁻¹) in 82 breast tumor specimens, and 1.07×10⁻³±1.62×10⁻³ (range, 0−4.86×10⁻³) in 17 normal/benign fibrocystic breast specimens (FIG. 5). All RNA derived from PEAT specimens had good quality and integrity for analysis. Relative B7-H3 mRNA copy number was significantly higher in breast cancer tissues than in normal/benign fibrocystic breast tissues (P=0.0029). Using the optimized qRT-PCR assay, B7-H3 mRNA was identified in 32 of 82 (39%) primary breast tumors (FIG. 5). The studies demonstrated that B7-H3 was expressed in a subset of primary breast cancers only, suggesting that there may be a potential pathobiology difference for individual primary tumors.

Primary Tumor B7-H3 mRNA Expression and Clinicopathological Factors

The potential role of B7-H3 expression was examined as a tumor progression factor. To determine the significance of B7-H3 expression by the primary tumor as a progression factor, its correlation to known breast cancer prognostic factors were assessed. The correlation between B7-H3 expression and clinicopathological factors, excluding lymph node metastasis status, was assessed (Table 1). B7-H3 mRNA expression significantly correlated only with primary tumor size (T stage), overall AJCC stage, and lymphovascular invasion (LVI) (P<0.0001, P<0.0001, and P=0.0071, respectively) as shown in Table 1. This suggested B7-H3 expression is related to early stages of progression of primary breast tumor.

TABLE 1
Primary Tumor B7-H3 mRNA Expression
and Clinicopathological Factors

| | | B7-H3 mRNA expression | | |
|---|---|---|---|---|
| | | Negative | Positive | P |
| Clinicopathological Factors | | n = 50 (%) | n = 32 (%) | value |
| Age (y) | ≦50 | 12 (24) | 8 (25) | 0.92 |
| | >50 | 38 (76) | 24 (75) | |
| Primary tumor size | ≦2 cm (T1) | 27 (54) | 1 (3) | <0.0001 |
| | >2 cm | | | |
| | and ≦5 cm | | | |
| (T stage) | (T2) | 14 (28) | 8 (25) | |
| | >5 cm(T3) | 9 (18) | 23 (72) | |
| AJCC stage | I | 18 (36) | 1 (3) | <0.0001 |
| | II | 17 (34) | 5 (16) | |
| | III | 15 (30) | 26 (81) | |
| Histologic grade | Well | 11 (22) | 10 (31) | 0.64 |
| | Moderate | 19 (38) | 11 (34) | |
| | Poor | 20 (40) | 11 (34) | |
| Histologic type | Ductal | 38 (76) | 19 (59) | 0.26 |
| | Lobular | 11 (22) | 12 (37) | |
| | Mucinous | 1 (2) | 1 (3) | |
| Estrogen receptor | Negative | 9 (18) | 4 (13) | 0.51 |
| | Positive | 41 (82) | 28 (88) | |
| Progesterone | Negative | 17 (34) | 16 (50) | 0.15 |
| receptor | Positive | 33 (66) | 16 (50) | |
| HER2/neu receptor | Negative | 40 (80) | 28 (88) | 0.38 |
| | Positive | 10 (20) | 4 (13) | |
| Lymphovascular | Absent | 38 (76) | 15 (47) | 0.007 |
| invasion | Present | 12 (24) | 17 (53) | |
| DNA ploidy | Diploid | 25 (50) | 18 (56) | 0.64 |
| | Aneuploid/ | 24 (48) | 14 (44) | |
| | tetraploid | | | |
| | Unknown | 1 (2) | 0 | |
| S-phase | Low | 21 (42) | 11 (34) | 0.36 |
| | Intermediate | 11 (22) | 11 (34) | |
| | High | 14 (28) | 6 (19) | |
| | Unknown | 4 (8) | 4 (13) | |
| Ki-67 | Low | 26 (52) | 17 (53) | 0.90 |
| | Intermediate | 13 (26) | 7 (22) | |
| | High | 11 (22) | 8 (25) | |
| p53 | Negative | 40 (80) | 27 (84) | 0.75 |
| | Positive | 9 (18) | 5 (16) | |
| | Unknown | 1 (2) | 0 | |

B7-H3 protein expression by IHC in primary tumors

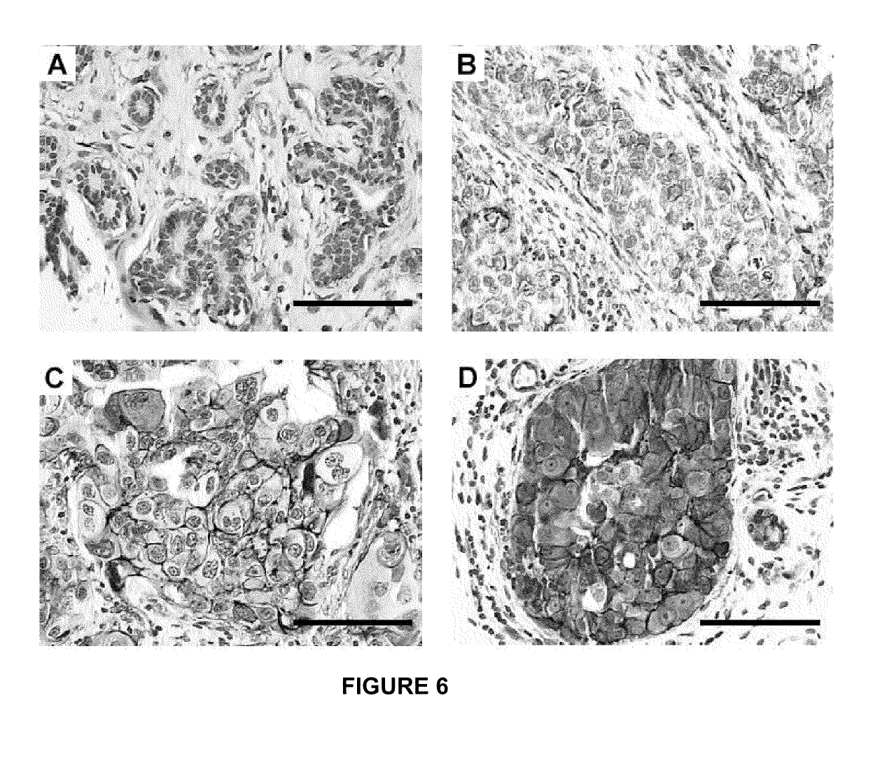

For assessment of B7-H3 protein expression IHC was performed on the same PEAT specimens that were used in the qRT-PCR assay. Twenty primary breast tumor specimens which varied according to B7-H3 mRNA expression were selected and immunostained; normal/benign fibrocystic breast specimens were evaluated as negative controls. B7-H3 protein expression was found in the cell membrane and/or cytoplasm of breast tumor cells. The normal breast specimens demonstrated negative or very weak background immunostaining (FIG. 6A). In contrast, B7-H3 protein expression levels varied in tumor specimens from strong to moderate immunostaining (FIG. 6B-D). The moderate or strong immunostaining for B7-H3 protein expression was observed in 9 of 10 (90%) tumors positive for B7-H3 mRNA expression (Table 3). Ten primary tumors were selected, respectively, from negative (n=50) and positive (n=32) for B7-H3 mRNA expression and assessed by B7-H3 IHC. B7-H3 IHC correlated with mRNA expression (P=0.0047). These results confirmed that B7-H3 mRNA expression levels were concordant with protein expression. The results also indicated B7-H3 is expressed in the cytoplasm and cell surface of breast cancer cells.

TABLE 3
Relationship Between B7-H3 mRNA and Protein Expression

| | | | | | Protein |
|---|---|---|---|---|---|
| Patients | AJCC | N | mRNA expression | | expression |
| Tumor | stage | stage | Relative mRNA copies | Status | IHC |
| 1 | II | N1 | 7.0 × 10−5 | Negative | + |
| 2 | III | N2 | 8.3 × 10−5 | Negative | ++ |
| 3 | II | N1 | 9.4 × 10−5 | Negative | + |
| 4 | II | N1 | 1.1 × 10−4 | Negative | + |
| 5 | I | N0 | 2.4 × 10−4 | Negative | + |
| 6 | I | N0 | 3.8 × 10−4 | Negative | ++ |
| 7 | II | N1 | 4.4 × 10−4 | Negative | + |
| 8 | I | N0 | 5.1 × 10−4 | Negative | + |
| 9 | II | N1 | 8.4 × 10−4 | Negative | + |
| 10 | I | N0 | 9.0 × 10−4 | Negative | + |
| 11 | II | N0 | 8.8 × 10−3 | Positive | ++ |
| 12 | II | N0 | 9.2 × 10−3 | Positive | + |
| 13 | III | N3 | 9.3 × 10−3 | Positive | ++ |
| 14 | III | N2 | 1.3 × 10−2 | Positive | ++ |
| 15 | III | N1 | 1.8 × 10−2 | Positive | ++ |
| 16 | III | N3 | 2.1 × 10−2 | Positive | +++ |
| 17 | III | N3 | 2.5 × 10−2 | Positive | +++ |
| 18 | III | N1 | 2.6 × 10−2 | Positive | +++ |
| 19 | III | N2 | 3.5 × 10−2 | Positive | +++ |
| 20 | III | N2 | 4.1 × 10−2 | Positive | +++ |

*Status refers to positive or negative

### Example 4

### B7-H3 Expression in Primary Breast Cancer Tumor Specimens is a Prognostic Factor for Lymph Node Status

Next, it was determined whether B7-H3 (+) primary breast cancers were associated with presence of regional lymph node metastasis. To date there is no single predictive factor to determine likelihood of regional metastasis when diagnosed with primary breast cancer. To assess the correlation between B7-H3 mRNA expression and of the presence/extent of axillary lymph node metastasis, all 82 patients from Example 4 above were divided into three groups based on the number of metastatic lymph nodes detected immediately after primary tumor diagnosis (0 versus 1 versus ≧2; Table 2A). Number of metastatic lymph nodes is an important known breast cancer prognostic factor in early stage disease. B7-H3 expression by primary tumors significantly correlated with an increase in the number of lymph nodes with metastasis (P=0.003). According to this analysis, B7-H3 primary tumor expression was positive in 58% of patients with ≧2 tumor-positive lymph nodes, as compared to only 17% of patients without lymph node metastasis.

TABLE 2A
Primary Tumor B7-H3 mRNA Expression and Lymph Node Status:
Number of lymph nodes with metastasis (0 vs 1 vs ≧2)

| Primary | Number of metastatic lymph nodes | | | | |
|---|---|---|---|---|---|
| Tumor | 0 | 1 | ≧2 | Total | |
| B7-H3 | n = 29 | n = 17 | n = 36 | n = 82 | P |
| expression | (%) | (%) | (%) | (%) | value |
| Negative | 24 (83) | 11 (65) | 15 (42) | 50 (61) | 0.0031 |
| Positive | 5 (17) | 6 (35) | 21 (58) | 32 (39) | |

When B7-H3 primary tumor expression was examined according to AJCC categories for the number of metastatic lymph nodes (0 versus 1-3 versus ≧4; Table 2B), B7-H3 expression rate increased with the number of metastatic lymph nodes (P=0.0035). B7-H3 expression was positive in 61% of patients with ≧4 metastatic lymph nodes.

TABLE 2B
Primary Tumor B7-H3 mRNA Expression and Lymph Node Status:
Number of lymph nodes with metastasis (0 vs 1-3 vs ≧4)

| Primary | Number of metastatic lymph nodes | | | | |
|---|---|---|---|---|---|
| Tumor | 0 | 1-3 | ≧4 | Total | |
| B7-H3 | n = 29 | n = 25 | n = 28 | n = 82 | P |
| expression | (%) | (%) | (%) | (%) | value |
| Negative | 24 (83) | 15 (60) | 11 (39) | 50 (61) | 0.0035 |
| Positive | 5 (17) | 10 (40) | 17 (61) | 32 (39) | |

The SLN mapping and lymphadenectomy procedure along with histopathology analysis that was previously developed (Giuliano et al. 1994; Morton et al. 1991) is considered the most accurate procedure to identify early stage breast cancer regional node micrometastasis (Giuliano et al. 1994; Olson et al. 2008; Turner et al. 2008). To evaluate the relation between B7-H3 mRNA expression and SLN status, 66 patients who underwent SLN lymphadenectomy were classified into 3 groups: node negative, SLN positive, or non-SLN (NSLN) positive as previously defined (Giuliano et al. 1994) (Table 2C). Patients with positive B7-H3 expression had significantly higher rates of SLN and NSLN metastasis than patients with negative B7-H3 expression (P=0.0025). B7-H3 mRNA expression was detected in 14 of 21 (67%) patients with NSLN metastasis. There was a significant relation between B7-H3 expression levels (continuous variable) and increasing burden of disease: N0 vs N1 vs N2 vs N3 (P=0.0195; Kruskal-Wallis Test).

TABLE 2C
Primary Tumor B7-H3 mRNA Expression
and Lymph Node Status: SLN status

| | SLN status | | | | |
|---|---|---|---|---|---|
| Primary | | | Non- | | |
| Tumor | SLN (−) | SLN (+) | SLN (+) | Total | |
| B7-H3 | n = 28 | n = 17 | n = 21 | n = 66 | P |
| expression | (%) | (%) | (%) | (%) | value |
| Negative | 23 (82) | 10 (59) | 7 (33) | 40 (61) | 0.0025 |
| Positive | 5 (18) | 7 (41) | 14 (67) | 26 (39) | |

66 patients underwent SLND. All patients with non-SLN (+) had metastasis in the SLN.

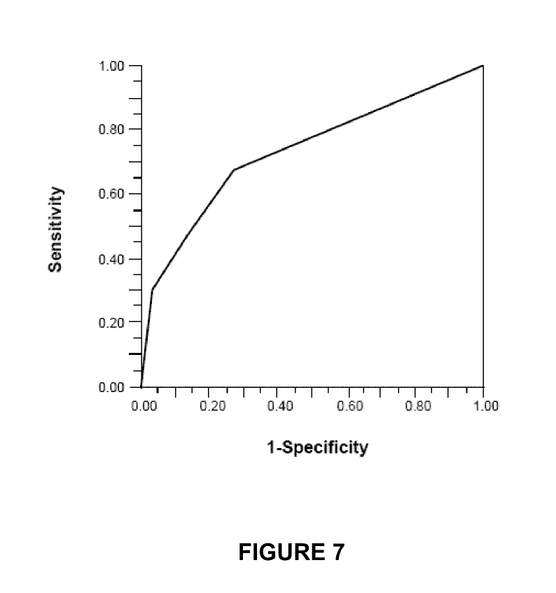

B7-H3 primary tumor expression and clinical factors that were significant in the univariate logistic analysis were included in a multivariate logistic regression analysis for the prediction of lymph node metastasis. In a multivariate analysis, LVI (OR, 4.246; 95% CI, 1.24-14.49, P=0.021), and B7-H3 expression (OR, 3.79; 95% CI, 1.20-11.95, P=0.023) were significantly correlated with lymph node metastasis. Based on the results of multivariate logistic regression analysis, ROC curves for the prediction of lymph node metastasis were constructed with reference to lymphovascular and B7-H3 expression. The AUC was 0.73 (FIG. 7). The values of sensitivity and specificity were 0.68 (95% CI, 0.54 to 0.80), and 0.72 (95% CI, 0.53 to 0.87), respectively. The positive predictive value was 0.82 and negative predictive value was 0.55 in the model system.

B7-H3 mRNA and Protein Expression in Metastatic Lymph Nodes

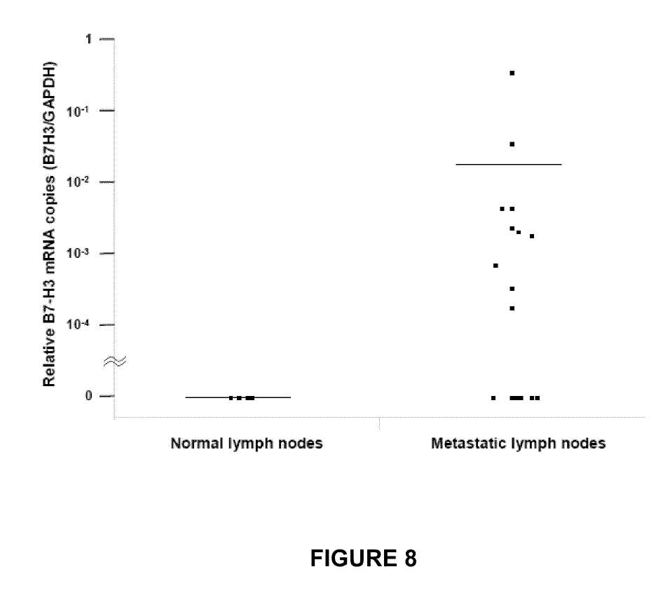

B7-H3 mRNA positivity was assessed by qRT assay in 24 PEAT tissues of metastatic SLNs and 10 PEAT tissues of tumor-free lymph nodes. Among the 24 patients with lymph node metastasis, the mean value of relative B7-H3 mRNA copies (±SD) was 1.77×10⁻²±7.56×10⁻² in 24 patients with SLNs metastasis (FIG. 8). On the other hand, B7-H3 was not detected in 10 histopathology tumor (−) SLN under optimal qRT conditions (P=0.019).

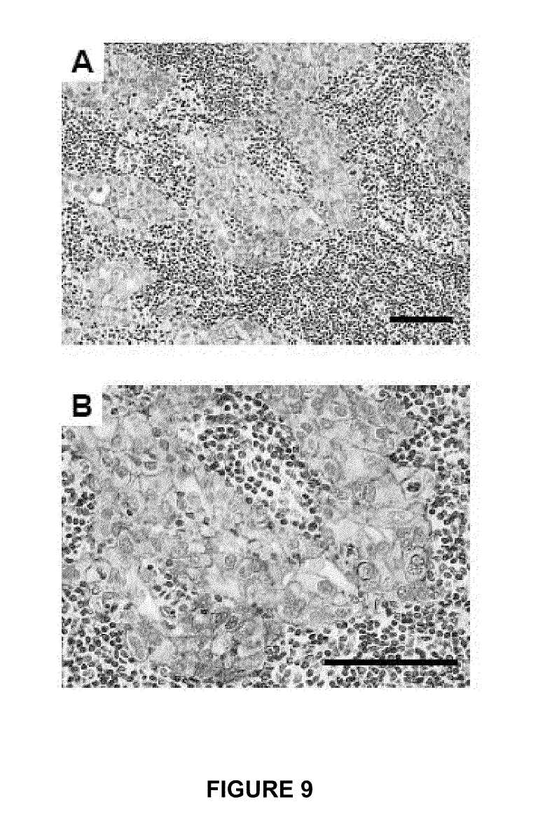

Furthermore, B7-H3 protein expression was assessed by IHC in 24 SLNs with metastasis. B7-H3 protein expression was detected in all SLNs with metastasis (FIGS. 9A, and 9B) and negative in tumor-free lymph nodes. In three distant lung metastases available, all were positive for B7-H3 expression.

### Example 5

### B7-H3 Antibody Coupled Bead Assay for Isolation and Detection of Circulating Tumor Cells

Circulating tumor cells may be isolated from the blood of metastatic and non-metastatic melanoma patients using a magnetic immunobead assay with B7-H3 monoclonal antibody (mAb). These B7-H3(+) CTC may also be evaluated for melanoma-associated biomarkers by quantitative real-time reverse transcription-polymerase chain reaction (qRT-PCR) assay (Goto et al. 2008; Koyanagi et al. 2005, Takeuchi et al. 2003) to verify the detection of isolated B7-H3(+) CTC. Verification may be accomplished using four known MAA qRT-PCR biomarkers (melanoma antigen recognized by T-cells-1 (MART-1), melanoma antigen gene-A3 family (MAGE-A3), high molecular weight-melanoma-associated antigen (HMW-MAA), and tyrosinase-related protein-2 (TRP-2)). The four melanoma biomarkers may also be used to further characterize the CTC. This assay should demonstrate that B7-H3 is expressed by cutaneous primary and metastatic melanomas and is related to tumor progression. Although the methods described below are directed to melanoma, similar methods may be used for other cancers with a high propensity of primary tumor metastasis to the lymph node such as breast cancer and gastrointestinal cancers such as gastric, colorectal, pancreatic and liver cancers.

Materials and Methods

Blood Specimens. Blood specimens may be collected from melanoma patients of AJCC stage I/II (n=8), III (n=35), and IV (n=11). Specimens of peripheral blood lymphocytes (PBLs) from healthy volunteers serve as a negative control group. All laboratory research investigators should be unaware of the disease status of patients. Informed human subject consent should be approved by the SJHC/John Wayne Cancer Institute Institutional Review Board, and should be obtained for specimens from all patients.

Blood processing and immunomagnetic isolation. 9 ml of blood from each patient is collected in 2×4.5 ml sodium citrate-containing tubes, and blood cells are processed using Purescript RBC Lysis Solution (Gentra Systems, Inc., Minneapolis, Minn.) as previously described (Koyanagi et al. 2005a). Peripheral blood cells (PBCs) are immediately incubated at 4° C. for 20 min with 2 μg of anti-B7-H3 mAb (R& D Systems, Minneapolis, Minn.). After washing in PBS with 0.1% bovine serum albumin, blood cells are incubated at 4° C. for 30 min with 25 μL of CELLection Pan Mouse IgG Dynabeads (Dynal, Invitrogen, CA). Bead-binding cells are isolated using a Dynal MPC-6 magnet (Dynal) and separated from beads with special releasing buffer (Dynal) in RPMI 1640 with 1% FBS as described by the manufacturer.

RNA extraction. Tri-Reagent (Molecular Research Center. Inc., Cincinnati, Ohio) may be used to extract total RNA from isolated cells as previously described (Koyanagi et al. 2005a, Koyanagi et al. 2006).

Primers and probes. Primer and probe sequences were designed for qRT-PCR assay as previously described (Goto et al. 2008; Koyanagi et al. 2005a; Takeuchi et al. 2003). The fluorescence resonance energy transfer probe sequences for the detection of CTC are as follows:

MART-1:
(SEQ ID NO: 5)
5′-FAM-CAGAACAGTCACCACCACCTTATT-BHQ-1-3′
MAGE-A3:
(SEQ ID NO: 6)
5′-FAM-AGCTCCTGCCCACACTCCCGCCTGT-BHQ-1-3′
HMW-MAA:
(SEQ ID NO: 7)
5′-FAM-AGGATCACCGTGGCTGCTCT-BHQ-1-3′
TRP-2:
(SEQ ID NO: 8)
5′-FAM-TCACATCAAGGACCTGCATTTGTTA-BHQ-1-3′
GAPDH:
(SEQ ID NO: 4)
5′-FAM-CAGCAATGCCTCCTGCACCACCAA-BHQ-1-3′

The GAPDH housekeeping gene is used as an internal control to confirm RNA integrity.

Primer and probe sequences of B7-H3 were designed to assess B7-H3 mRNA expression in CTC isolated from blood of melanoma patients. The forward primer, fluorescence resonance energy transfer probe sequence, and reverse primer for B7-H3 are as follows:

B7-H3 forward:
(SEQ ID NO: 1)
5′-GACAGCAAAGAAGATGATGGA-3′
B7-H3 probe:
(SEQ ID NO: 2)
5′-FAM-CCTCCCTACAGCTCCTACCCTCTGG-BHQ-1-3′
B7-H3 reverse:
(SEQ ID NO: 3)
5′-ACCTGTCAGAGCAGGATGC-3′

qRT-PCR assay. All reverse transcription reactions of total RNA may be done using Moloney murine leukemia virus reverse transcriptase (Promega, Madison, Wis.), oligo-dT primer (Gene Link, Hawthorne, N.Y.) and random hexamers (Roche Diagnostics, Indianapolis, Ind.) as previously described (Koyanagi et al. 2005a; Nakagawa et al. 2007). The qRT-PCR assay may be performed with the iCycler iQ Real-Time Thermocycler Detection System (Bio-Rad Laboratories, Hercules, Calif.) as previously described (Koyanagi et al. 2005a; Nakagawa et al. 2007).

For each reaction, complementary DNA from 250 ng of RNA may be used with a reaction mixture containing each primer, probe, and iTaq custom Supermix (Bio-Rad). Samples may be amplified with a precycling hold at 95° C. for 10 min, followed by optimized cycles of denaturation for each marker at 95° C. for 60 sec (40 cycles for MART-1, MAGE-A3, GAPDH, and B7-H3; 37 cycles for HMW-MAA; and 35 cycles for TRP-2), annealing for 60 sec (59° C. for MART-1; 58° C. for MAGE-A3; 63° C. for HMW-MAA and B7-H3; and 55° C. for TRP-2 and GAPDH), and extension at 72° C. for 60 sec. Specific plasmids for external controls of each marker may be synthesized as described previously (Takeuchi et al. 2003). Standard curves for each assay may be generated using a threshold cycle of six serial dilutions of plasmid templates (10⁶-10¹ copies), and the mRNA copy number can be calculated using the iCycler iQ Real-Time Thermocycler Detection System Software (Bio-Rad). Each assay should be repeated in duplicate with positive (ME-2) and reagent controls (reagent alone) for verification of the qRT-PCR assay.

Sensitivity of immunomagnetic isolation. To assess the detectable limit and clinical feasibility of the multimarker qRT-PCR assay combined with immunomagnetic bead isolation, the sensitivity of this assay may be tested by spiking 10-fold dilutions of ME-2 cells (10⁴, 10³, 10², 10¹, and 0) into 5×10⁶ PBCs isolated from healthy volunteer donor samples' blood.

### Example 6

### Detection of B7-H3 in Circulating Tumor Cells by a Direct qRT-PCR Assay

The level of B7-H3 expression in circulating blood cells or in other bodily fluids (e.g., cerebrospinal fluid (CSF), spinal fluid, synovial fluid, ascetic fluid, pericardial fluid and peritoneal fluid) can be determined by a direct qRT-PCR assay as described below. This level may serve as a metastasis predictor directly or as a method for detecting CTCs in the blood or bodily fluid.

Materials and Methods

Patient Specimens. Blood specimens (or other bodily fluid specimens) may be collected from cancer patients of AJCC stage I/II, Ill, and IV. Specimens of peripheral blood lymphocytes (PBLs) from healthy volunteers should be collected to serve as a negative control group. All laboratory research investigators should be unaware of the disease status of patients. Informed human subject consent should be approved by the SJHC/John Wayne Cancer Institute Institutional Review Board, and should be obtained for specimens from all patients.

Blood processing and immunomagnetic isolation. 9 ml of blood from each patient is collected in 2×4.5 ml sodium citrate-containing tubes, and blood cells are processed using Purescript RBC Lysis Solution (Gentra Systems, Inc., Minneapolis, Minn.) as previously described (Koyanagi et al. 2005a). Peripheral blood cells (PBCs) are immediately incubated at 4° C. for 20 min with 2 μg of anti-B7-H3 mAb (R& D Systems, Minneapolis, Minn.). After washing in PBS with 0.1% bovine serum albumin, blood cells are incubated at 4° C. for 30 min with 25 μL of CELLection Pan Mouse IgG Dynabeads (Dynal, Invitrogen, CA). Bead-binding cells are isolated using a Dynal MPC-6 magnet (Dynal) and separated from beads with special releasing buffer (Dynal) in RPMI 1640 with 1% FBS as described by the manufacturer.

RNA extraction. Tri-Reagent (Molecular Research Center. Inc., Cincinnati, Ohio) was used to extract total RNA from isolated cells as previously described (Koyanagi et al. 2005a, Koyanagi et al. 2006).

Primers and probes. Primer and probe sequences were designed for qRT-PCR assay as previously described (Goto et al. 2008; Koyanagi et al. 2005a; Takeuchi et al. 2003). Primer and probe sequences of B7-H3 (B7-H3-variant-middle-141-F) were designed to assess B7-H3 mRNA expression in CTC isolated from blood of melanoma patients, but can be designed for any type of metastatic cancer. The forward primer, fluorescence resonance energy transfer probe sequence, and reverse primer for B7-H3-variant-middle-141-F are as follows:

B7-H3-variant forward:
(SEQ ID NO: 9)
5′-AGAGAAGCCCCACAGGAG-3′
B7-H3-variant probe:
(SEQ ID NO: 10)
5′-FAM-GAGGTCCAGGTCCCTGAGGACC-BHQ-1-3′
B7-H3-variant reverse:
(SEQ ID NO: 11)
5′-GCTGCCAGATGAGGTTGA-3′

In addition, the GAPDH housekeeping gene is used as an internal control to confirm RNA integrity:

GAPDH probe:
(SEQ ID NO: 4)
5′-FAM-CAGCAATGCCTCCTGCACCACCAA-BHQ-1-3′

qRT-PCR assay. All reverse transcription reactions of total RNA may be done using Moloney murine leukemia virus reverse transcriptase (Promega, Madison, Wis.), oligo-dT primer (Gene Link, Hawthorne, N.Y.) and random hexamers (Roche Diagnostics, Indianapolis, Ind.) as previously described (Koyanagi et al. 2005a; Nakagawa et al. 2007). The qRT-PCR assay can be performed with the iCycler iQ Real-Time Thermocycler Detection System (Bio-Rad Laboratories, Hercules, Calif.) as previously described (Koyanagi et al. 2005a; Nakagawa et al. 2007).

For each reaction, complementary DNA from 250 ng of RNA may be used with a reaction mixture containing each primer, probe, and iTaq custom Supermix (Bio-Rad). Samples may be amplified by cycles of 50° C. for 2 minutes, 95° C. for 10 minutes, followed by 45 cycles of denaturing at 95° C. for 15 seconds, 1 minute annealing at 55° C. for GAPDH, and at 60° C. for B7-H3 using an Applied BioSystems ABI-7900HT Real-Time PCR Detection System for qRT-PCR. Positive (melanoma cell lines), negative (normal blood) and reagent controls (without RNA or cDNA) should be included in each qRT-PCR assay and each assay should be run in triplicates for verification and the Ct (threshold) values used for data analysis.

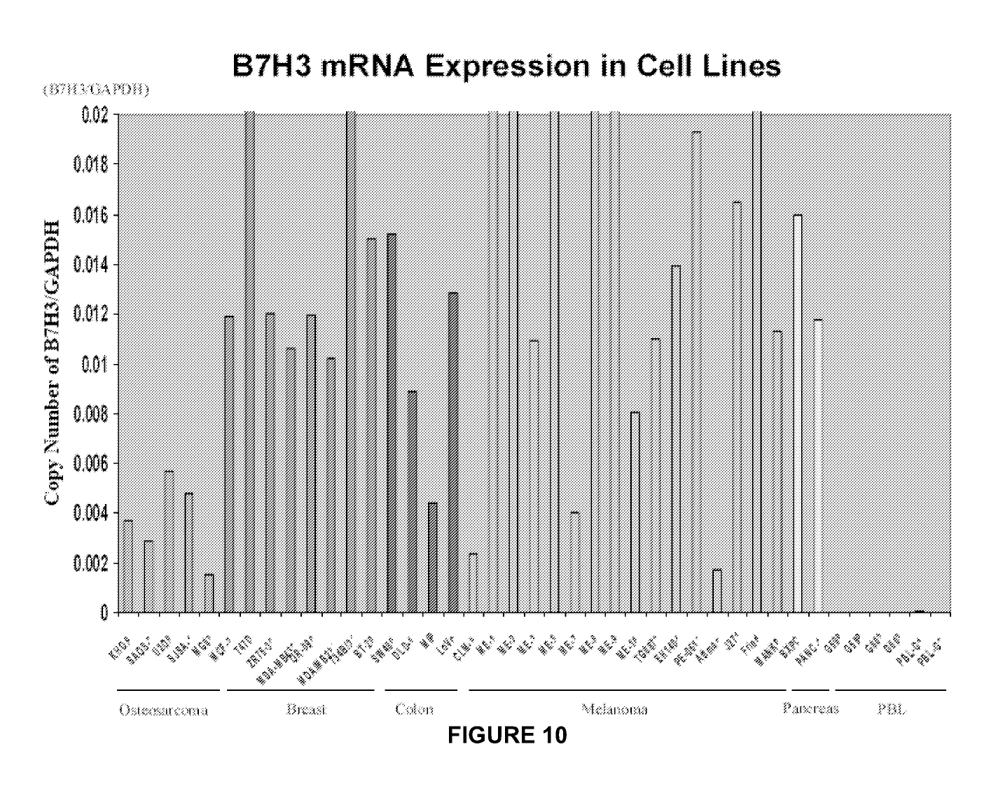

Results of the Direct qRT-PCR assay are expressed in relative copy number of B7-H3 to GAPDH. FIG. 10 shows exemplar results of the Direct qRT-PCR assay showing high levels of B7-H3 in several cancer cell lines.

### Example 7

### B7-H3 is an Efficient Cell Surface Marker for Detecting Metastatic Osteosarcoma Cells

As shown below, B7-H3 is also present on sarcoma (osteosarcoma is shown here) cells. Thus. B7-H3 may also be used to detect circulating tumor cells in sarcoma patients.

Materials and Methods

Osteosarcoma cell lines. Five established human osteosarcoma cell lines (U-2-OS, SJSA-1, Saos-2, MG-63 and KHOS/NP) were used in this study. These osteosarcoma lines were cultured in GIBCO RPMI 1640 (Invitrogen, Carlsbad, Calif.), and supplemented with 10% heat-inactivated fetal bovine serum (FBS), 100 units/mL penicillin, and 100 units/mL streptomycin. All cell lines were grown at 37° C. in a humidified atmosphere at 5% CO₂ as previously described (Nakagawa et al. 2007). Melanocyte primary cultures were commercially (Lonza Inc., Allendale, N.J.) obtained and grown in specific tissue culture medium as suggested by the manufacturers.

Flow cytometry. Flow cytometric analysis was performed using the BD FACSCalibur System (BD Biosciences, San Jose, Calif.). After washing in flow cytometry buffer (PBS with 1% FBS) to block nonspecific binding, 1×10⁶ osteosarcoma cells were incubated at 4° C. for 1 hr with 1 μg of anti-B7-H3 mAb (R& D Systems, Minneapolis, Minn.). These cells were stained at 4° C. for 30 min with an optimal amount of phycoerythrin (PE)-labeled F(ab′)₂ fragment of goat anti-mouse IgG (Santa Cruz Biotechnology) after washing in flow cytometry buffer. Cells were fixed in 4% formaldehyde and analyzed using Cell Quest software (Becton Dickinson, Franklin Lakes, N.J.). Isotype-matched antibodies were used as negative controls.

Flow Cytometric Analysis of B7-H3 Expression.

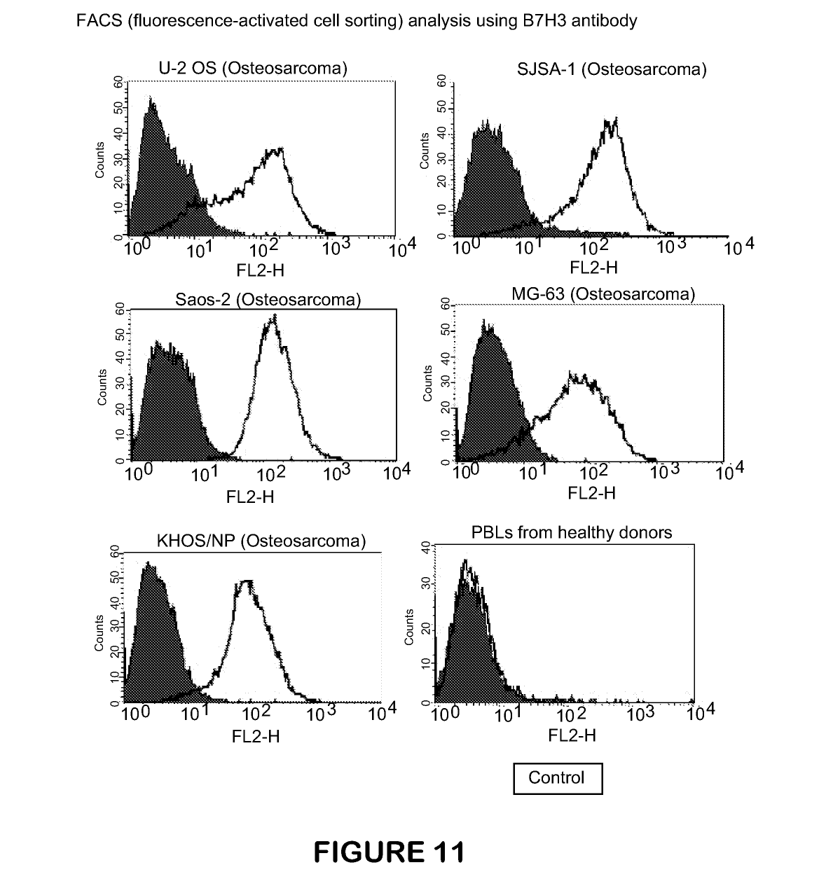

Osteosarcoma lines U-2-OS, SJSA-1, Saos-2, MG-63 and KHOS/NP, along with PBCs from healthy volunteers as negative controls, were assessed to determine whether B7-H3 is a good marker for determining the presence of metastatic osteosarcoma cells. Flow cytometric analysis demonstrated that B7-H3 was highly expressed on the cell surface of all melanoma cell lines (n=5) and not on normal donor PBCs as shown in FIG. 11 (B7-H3 expression shown by open histogram).

### REFERENCES

The references listed below, and all references cited in the specification are hereby incorporated by reference in their entireties, as if fully set forth herein.
Azuma M, Ito D, Yagita H, et al. B70 antigen is a second ligand for CTLA-4 and CD28. Nature 1993; 366(6450):76-9.
Balch C M, Buzaid A C, Soong S J, et al. Final version of the American Joint Committee on Cancer staging system for cutaneous melanoma. J Clin Oncol 2001; 19:3635-48.
Beck K E, Blansfield J A, Tran K Q, et al. Enterocolitis in patients with cancer after antibody blockade of cytotoxic T-lymphocyte-associated antigen 4. J Clin Oncol 2006; 24(15):2283-9.
Bruland O S, Hoifodt H, Saeter G, Smeland S, and Fodstad O. Hematogenous micrometastases in osteosarcoma patients. Clin Cancer Res 2005; 11:4666-73.
Brunet J F, Denizot F, Luciani M F, et al. A new member of the immunoglobulin superfamily—CTLA-4. Nature 1987; 328(6127):267-70.
Castriconi R, Dondero A, Augugliaro R, et al. Identification of 4Ig-B7-H3 as a neuroblastoma-associated molecule that exerts a protective role from an NK cell-mediated lysis. Proc Natl Acad Sci USA 2004; 101:12640-5.
Chapoval Al, Ni J, Lau J S, et al. B7-H3: a costimulatory molecule for T cell activation and IFN-gamma production. Nat Immunol 2001; 2:269-74.
Chen L. Co-inhibitory molecules of the B7-CD28 family in the control of T-cell immunity. Nat Rev Immunol 2004; 4(5):336-47.
Chen Y W, Tekle C, and Fodstad 0. The immunoregulatory protein human B7H3 is a tumor-associated antigen that regulates tumor cell migration and invasion. Curr Cancer Drug Targets 2008; 8:404-13.
Crispen P L, Sheinin Y, Roth T J, et al. Tumor cell and tumor vasculature expression of B7-H3 predict survival in clear cell renal cell carcinoma. Clin Cancer Res 2008; 14(16):5150-7.
Dong H, Strome S E, Salomao D R, et al. Tumor-associated B7-H1 promotes T-cell apoptosis: a potential mechanism of immune evasion. Nat Med 2002; 8(8):793-800.
Faye R S, Aamdal S, Hoifodt H K, et al. Immunomagnetic detection and clinical significance of micrometastatic tumor cells in malignant melanoma patients. Clin Cancer Res 2004; 10:4134-9.
Flatmark K, Bjornland K, Johannessen H O, et al. Immunomagnetic detection of micrometastatic cells in bone marrow of colorectal cancer patients. Clin Cancer Res 2002; 8:444-9.
Flies D B and Chen L. The new B7s: playing a pivotal role in tumor immunity. J Immunother 2007; 30:251-60.
Freeman G J, Gribben J G, Boussiotis V A, et al. Cloning of B7-2: a CTLA-4 counter-receptor that costimulates human T cell proliferation. Science 1993; 262(5135):909-11.
Fieger C, Chen F, Coberly S, et al. The anti-B7-H3-4Ig antibody TES7 recognizes cancer stem cell lines, modulates angiogenic factor secretion, and exhibits potent anti-tumor activity in vivo [abstract]. In: Proceedings of the 99th Annual Meeting of the American Association for Cancer Research; Apr. 12-16, 2008; San Diego, Calif. Abstract #2555
Giuliano A E, Kirgan D M, Guenther J M, Morton D L. Lymphatic mapping and sentinel lymphadenectomy for breast cancer. Ann Surg 1994; 220(3):391-8; discussion 398-401.
Goto Y, Arigami T, Kitago M, et al. Activation of Toll-like receptors 2, 3, and 4 on human melanoma cells induces inflammatory factors. Mol Cancer Ther 2008; 7:3642-53.
Goto Y, Ferrone S, Arigami T, et al. Human high molecular weight-melanoma-associated antigen: utility for detection of metastatic melanoma in sentinel lymph nodes. Clin Cancer Res 2008; 14:3401-7.
Goto Y, Matsuzaki Y, Kurihara S, et al. A new melanoma antigen fatty acid-binding protein 7, involved in proliferation and invasion, is a potential target for immunotherapy and molecular target therapy. Cancer Res 2006; 66(8):4443-9.
Greenwald R J, Freeman G J, and Sharpe A H. The B7 family revisited. Annu Rev Immunol 2005; 23:515-48.
Hashiguchi M, Kobori H, Ritprajak P, et al. Triggering receptor expressed on myeloid cell-like transcript 2 (TLT-2) is a counter-receptor for B7-H3 and enhances T cell responses. Proc Natl Acad Sci USA 2008; 105(30):10495-500.
Hodi F S, Mihm M C, Soiffer R J, et al. Biologic activity of cytotoxic T lymphocyte-associated antigen 4 antibody blockade in previously vaccinated metastatic melanoma and ovarian carcinoma patients. Proc Natl Acad Sci USA 2003; 100(8):4712-7.
Hofmeyer K A, Ray A, and Zang X. The contrasting role of B7-H3. Proc Natl Acad Sci USA 2008; 105:10277-8.
Hoon D S, Korn E L, Cochran A J. Variations in functional immunocompetence of individual tumor-draining lymph nodes in humans. Cancer Res 1987a; 47(6):1740-4.
Hoon D S, Bowker R J, Cochran A J. Suppressor cell activity in melanoma-draining lymph nodes. Cancer Res 1987b; 47(6):1529-33.
Hoon D S, Bostick P, Kuo C, et al. Molecular markers in blood as surrogate prognostic indicators of melanoma recurrence. Cancer Res 2000; 60:2253-7.
Hoon D S, Wang Y, Dale P S, et al. Detection of occult melanoma cells in blood with a multiple-marker polymerase chain reaction assay. J Clin Oncol 1995; 13:2109-16.
Hashiguchi M, Kobori H, Ritprajak P, Kamimura Y, Kozono H, and Azuma M. Triggering receptor expressed on myeloid cell-like transcript 2 (TLT-2) is a counter-receptor for B7-H3 and enhances T cell responses. Proc Natl Acad Sci USA 2008; 105:10495-500.
Imai K, Wilson B S, Bigotti A, Natali P G, and Ferrone S. A 94,000-dalton glycoprotein expressed by human melanoma and carcinoma cells. J Natl Cancer Inst 1982; 68:761-9.
Kielhorn E, Schofield K, and Rimm D L. Use of magnetic enrichment for detection of carcinoma cells in fluid specimens. Cancer 2002; 94:205-11.
Kim J, Takeuchi H, Lam S T, et al. Chemokine receptor CXCR4 expression in colorectal cancer patients increases the risk for recurrence and for poor survival. J Clin Oncol 2005; 23(12):2744-53.
Kim J, Reber H A, Hines O J, et al. The clinical significance of MAGEA3 expression in pancreatic cancer. Int J Cancer 2006; 118:2269-75.
Koyanagi K, Kuo C, Nakagawa T, et al. Multimarker quantitative real-time PCR detection of circulating melanoma cells in peripheral blood: relation to disease stage in melanoma patients. Clin Chem 2005a; 51:981-8.
Koyanagi K, O'Day S J, Gonzalez R, et al. Serial monitoring of circulating melanoma cells during neoadjuvant biochemotherapy for stage III melanoma: outcome prediction in a multicenter trial. J Clin Oncol 2005b; 23:8057-64.
Koyanagi K, O'Day S J, Gonzalez R, et al. Serial monitoring of circulating melanoma cells during neoadjuvant biochemotherapy for stage III melanoma: outcome prediction in a multicenter trial. J Clin Oncol 2005c; 23(31):8057-64.
Koyanagi K, O'Day S J, Gonzalez R, et al. Microphthalmia transcription factor as a molecular marker for circulating tumor cell detection in blood of melanoma patients. Clin Cancer Res 2006; 12:1137-43.
Ling V, Wu P W, Spaulding V, et al. Duplication of primate and rodent B7-H3 immunoglobulin V- and C-like domains: divergent history of functional redundancy and exon loss. Genomics 2003; 82(3):365-77.
Linsley P S, Brady W, Urnes M, et al. CTLA-4 is a second receptor for the B cell activation antigen B7. J Exp Med 1991; 174(3):561-9.
McShane L M, Altman D G, Sauerbrei W, et al. Reporting recommendations for tumor marker prognostic studies (REMARK). J Natl Cancer Inst 2005; 97(16):1180-4.
Mocellin S, Hoon D, Ambrosi A, Nitti D, and Rossi C R. The prognostic value of circulating tumor cells in patients with melanoma: a systematic review and meta-analysis. Clin Cancer Res 2006; 12:4605-13.
Morton D L, Wanek L, Nizze J A, et al. Improved long-term survival after lymphadenectomy of melanoma metastatic to regional nodes. Analysis of prognostic factors in 1134 patients from the John Wayne Cancer Clinic. Ann Surg 1991; 214(4):491-9; discussion 499-501.
Nakagawa T, Martinez S R, Goto Y, et al. Detection of circulating tumor cells in early-stage breast cancer metastasis to axillary lymph nodes. Clin Cancer Res 2007; 13:4105-10.
O'Mahony D, Morris J C, Quinn C, et al. A pilot study of CTLA-4 blockade after cancer vaccine failure in patients with advanced malignancy. Clin Cancer Res 2007; 13(3):958-64.
O'Day S J, Hamid O, and Urba W J. Targeting cytotoxic T-lymphocyte antigen-4 (CTLA-4): a novel strategy for the treatment of melanoma and other malignancies. Cancer 2007; 110:2614-27.
Olson J A, Jr., McCall L M, Beitsch P, et al. Impact of immediate versus delayed axillary node dissection on surgical outcomes in breast cancer patients with positive sentinel nodes: results from American College of Surgeons Oncology Group Trials Z0010 and Z0011. J Clin Oncol 2008; 26(21):3530-5.
Prasad D V, Nguyen T, Li Z, et al. Murine B7-H3 is a negative regulator of T cells. J Immunol 2004; 173(4):2500-6.
Ribas A, Camacho L H, Lopez-Berestein G, et al. Antitumor activity in melanoma and anti-self responses in a phase I trial with the anti-cytotoxic T lymphocyte-associated antigen 4 monoclonal antibody CP-675,206. J Clin Oncol 2005; 23(35):8968-77.
Rosenberg C D, Ferrone S, Hamby C V, Mancino V, and Graf L H, Jr. Interspecific DNA-mediated transfer and amplification of a gene specifying a Mr 100,000 human melanoma-associated cell surface glycoprotein. Cancer Res 1990; 50:1559-65.
Roth T J, Sheinin Y, Lohse C M, et al. B7-H3 ligand expression by prostate cancer: a novel marker of prognosis and potential target for therapy. Cancer Res 2007; 67:7893-900.
Sharpe A H and Freeman G J. The B7-CD28 superfamily. Nat Rev Immunol 2002; 2:116-26.
Singletary S E, Connolly J L. Breast cancer staging: working with the sixth edition of the AJCC Cancer Staging Manual. CA Cancer J Clin 2006; 56(1):37-47; quiz 50-1
Small E J, Tchekmedyian N S, Rini B I, Fong L, Lowy I, and Allison J P. A pilot trial of CTLA-4 blockade with human anti-CTLA-4 in patients with hormone-refractory prostate cancer. Clin Cancer Res 2007; 13:1810-5.
Suh W K, Gajewska B U, Okada H, et al. The B7 family member B7-H3 preferentially down-regulates T helper type 1-mediated immune responses. Nat Immunol 2003; 4(9):899-906.
Sun Y, Wang Y, Zhao J, et al. B7-H3 and B7-H4 expression in non-small-cell lung cancer. Lung Cancer 2006; 53:143-51.
Takeuchi H, Kuo C, Morton D L, et al. Expression of differentiation melanoma-associated antigen genes is associated with favorable disease outcome in advanced-stage melanomas. Cancer Res 2003; 63(2):441-8.
Takeuchi H, Morton D L, Kuo C, et al. Prognostic significance of molecular upstaging of paraffin-embedded sentinel lymph nodes in melanoma patients. J Clin Oncol 2004; 22(13):2671-80.
Takeuchi H, Kuo C, Morton D L, Wang H J, and Hoon D S. Expression of differentiation melanoma-associated antigen genes is associated with favorable disease outcome in advanced-stage melanomas. Cancer Res 2003; 63:441-8.
Thompson R H, Gillett M D, Cheville J C, et al. Costimulatory B7-H1 in renal cell carcinoma patients: Indicator of tumor aggressiveness and potential therapeutic target. Proc Natl Acad Sci USA 2004; 101(49):17174-9.
Tirapu I, Huarte E, Guiducci C, et al. Low surface expression of B7-1 (CD80) is an immunoescape mechanism of colon carcinoma. Cancer Res 2006; 66(4):2442-50.
Tivol E A, Borriello F, Schweitzer A N, et al. Loss of CTLA-4 leads to massive lymphoproliferation and fatal multiorgan tissue destruction, revealing a critical negative regulatory role of CTLA-4. Immunity 1995; 3(5):541-7.
Turner R R, Weaver D L, Cserni G, et al. Nodal stage classification for breast carcinoma: improving interobserver reproducibility through standardized histologic criteria and image-based training. J Clin Oncol 2008; 26(2):258-63.
Umetani N, Mori T, Koyanagi K, et al. Aberrant hypermethylation of ID4 gene promoter region increases risk of lymph node metastasis in T1 breast cancer. Oncogene 2005; 24:4721-7.
Umetani N, Takeuchi H, Fujimoto A, Shinozaki M, Bilchik A J, and Hoon D S. Epigenetic inactivation of ID4 in colorectal carcinomas correlates with poor differentiation and unfavorable prognosis. Clin Cancer Res 2004; 10:7475-83.
Waterhouse P, Penninger J M, Timms E, et al. Lymphoproliferative disorders with early lethality in mice deficient in Ctla-4. Science 1995; 270(5238):985-8.
Werther K, Normark M, Hansen B F, Brunner N, and Nielsen H J. The use of the CELLection kit in the isolation of carcinoma cells from mononuclear cell suspensions. J Immunol Methods 2000; 238:133-41.
Wu C P, Jiang J T, Tan M, et al. Relationship between co-stimulatory molecule B7-H3 expression and gastric carcinoma histology and prognosis. World J Gastroenterol 2006; 12:457-9.
Zang X, Allison J P. The B7 family and cancer therapy: costimulation and coinhibition. Clin Cancer Res 2007; 13(18 Pt 1):5271-9.
Zang X, Loke P, Kim J, Murphy K, Waitz R, and Allison J P. B7x: a widely expressed B7 family member that inhibits T cell activation. Proc Natl Acad Sci USA 2003; 100:10388-92.
Zang X and Allison J P. The B7 family and cancer therapy: costimulation and coinhibition. Clin Cancer Res 2007; 13:5271-9.

What is claimed is:

1. A method for diagnosing the progression of cancer with a high propensity of primary tumor metastasis to the lymph node or distant site comprising:
obtaining a cancer tissue sample from a cancer patient; and
determining an expression level of B7-H3 present in the tissue sample; and
diagnosing the progression of the cancer having a high propensity of primary tumor metastasis to the lymph node or distant site based upon the expression level, wherein an increased expression level correlates with an increased probability of having regional lymph nodes or organ site that are positive for metastases.

2. The method of claim 1, wherein the cancer tissue sample is a primary or metastatic tumor tissue specimen.

3. The method of claim 2, wherein the expression level of B7-H3 is determined by immunohistochemistry.

4. The method of claim 2, wherein the expression level of B7-H3 is determined by a direct qRT-PCR assay.

5. The method of claim 1, wherein the cancer tissue sample is a blood specimen from a cancer patient.

6. The method of claim 5, wherein the expression level of B7-H3 is determined by an anti-B7-H3 magnetic bead capture assay.

7. The method of claim 5, wherein the expression level of B7-H3 is determined by a direct qRT-PCR assay.

8. The method of claim 6 or 7, wherein the assay detects B7-H3 expressed on circulating tumor cells.

9. The method of claim 1, wherein the cancer is selected from the group consisting of melanoma, breast cancer, gastrointestinal cancers such as gastric cancer, colorectal, periampullary, pancreatic, liver cancer.

10. The method of claim 1, wherein the cancer is melanoma.

11. The method of claim 1, wherein the cancer is breast cancer.

12. A method for predicting nodal metastasis in breast cancer comprising:
obtaining a primary breast cancer tumor tissue sample from a breast cancer patient; and
determining an expression level of B7-H3 present in the primary breast cancer tumor tissue sample;
diagnosing progression of breast cancer wherein an increase in the B7-H3 expression level correlates with an increase in the number of lymph nodes having metastases.

13. The method of claim 12, further comprising: obtaining a lymph node tissue from a breast cancer patient; and determining the expression level of B7-H3 present in the lymph node.

14. The method of claim 12, wherein the expression level of B7-H3 is determined by immunohistochemistry.

15. The method of claim 12, wherein the expression level of B7-H3 is determined by qRT-PCR or any quantitative or binary molecular detection assays for B7-H3.

16. The method of claim 12, further predicting primary tumor size, wherein an increase in the B7-H3 expression level correlates with an increase in tumor size.

17. The method of claim 12, further predicting AJCC stage of breast cancer, wherein an increase in the B7-H3 expression correlates with a higher stage.

18. A method for predicting the progression of melanoma cancer comprising
obtaining a melanoma tumor tissue sample; and
determining an expression level of B7-H3 present in the melanoma tumor tissue sample; and
diagnosing progression of melanoma, wherein an increase in B7-H3 expression correlates with an increase in primary tumor tissue metastasis.

19. The method of claim 18, wherein the expression level of B7-H3 is determined by immunohistochemistry.

20. The method of claim 18, wherein the expression level of B7-H3 is determined by qRT-PCR, or any quantitative or binary molecular detection assays for B7-H3

21. A method for detecting circulating tumor cells in a cancer patient, comprising:
obtaining a bodily fluid sample from a cancer patient;
performing an assay to detect the presence of a B7-H3 expression level in the bodily fluid sample, wherein a higher expression level of B7-H3 correlates with a higher number of circulating tumor cells.

22. The method of claim 21, wherein the bodily fluid sample is selected from the group consisting of blood, cerebrospinal fluid (CSF), spinal fluid, synovial fluid, ascetic fluid, pericardial fluid and peritoneal fluid.

23. The method of claim 21, wherein the assay is qRT-PCR.

24. The method of claim 21, wherein the assay is an anti-B7-H3 magnetic bead capture assay.

25. A method for treating a cancer with a high propensity of primary tumor metastasis to the lymph node or distant site comprising:
obtaining a bodily fluid sample from a cancer patient;
detecting the presence of a population of B7-H3-positive CTC in the bodily fluid; and
administering a therapeutically effective amount of a pharmaceutical composition, the pharmaceutical composition comprising a B7-H3 ligand conjugated to a cytotoxic drug;
wherein the pharmaceutical composition kills B7-H3 circulating tumor cells and prevents metastasis.

26. The method of claim 25, wherein the bodily fluid is selected from the group consisting of blood, cerebrospinal fluid (CSF), spinal fluid, synovial fluid, ascetic fluid, pericardial fluid and peritoneal fluid.

27. The method of claim 25, wherein detection of a population of B7-H3-positive CTC is accomplished by performing qRT-PCR on the bodily fluid sample.

28. The method of claim 25, wherein detection of a population of B7-H3-positive CTC is accomplished by performing an anti-B7-H3 magnetic bead capture assay.

29. The method of claim 25, wherein the B7-H3 targeted therapy is a B7-H3 ligand conjugated with a cytotoxic drug.
